(19) United States
(12) Patent Application Publication
(10) Pub. No.: US 2010/0022476 A1
(43) Pub. Date: Jan. 28, 2010

(54) PYRAZOLYLAMINO SUBSTITUTED QUINAZOLES FOR THE TREATMENT OF CANCER

(75) Inventor: Kevin Michael Foote, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/722,439

(22) PCT Filed: Dec. 16, 2005
(86) PCT No.: PCT/GB2005/004872

§ 371 Date: Jun. 21, 2007

(30) Foreign Application Priority Data
Dec. 21, 2004 (GB) 0427917.0

(51) Int. Cl. A61K 31/675; A61P 35/00; C07D 401/12; A61K 31/517; A61K 31/496

(52) U.S. Cl. 514/80; 544/284; 514/266.23; 514/252.17

(57) ABSTRACT

The invention provided a compound of formula: (I) for use in the treatment of disease, in particular proliferative diseases such as cancer and for use in the preparation of medicaments for use in the treatment of proliferative diseases; the invention also processes for the preparation of such compounds, as well as pharmaceutical compositions containing them as active ingredient.

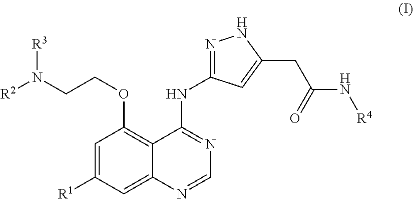

The present invention relates to quinazoline derivatives for use in the treatment of disease, in particular proliferative diseases such as cancer, in the preparation of medicaments for use in the treatment of proliferative diseases, and to processes for their preparation, as well as pharmaceutical compositions containing them as active ingredient.

Cancer (and other hyperproliferative diseases) are characterised by uncontrolled cellular proliferation. This loss of the normal regulation of cell proliferation often appears to occur as the result of genetic damage to cellular pathways that control progress through the cell cycle.

In eukaryotes, an ordered cascade of protein phosphorylation is thought to control the cell cycle. Several families of protein kinases that play critical roles in this cascade have now been identified. The activity of many of these kinases is increased in human tumours when compared to normal tissue. This can occur by either increased levels of expression of the protein (as a result of gene amplification for example), or by changes in expression of co activators or inhibitory proteins.

The first identified, and most widely studied of these cell cycle regulators have been the cyclin dependent kinases (or CDKs). Activity of specific CDKs at specific times is essential for both initiation and coordinated progress through the cell cycle. For example, the CDK4 protein appears to control entry into the cell cycle (the G0-G1-S transition) by phosphorylating the retinoblastoma gene product pRb. This stimulates the release of the transcription factor E2F from pRb, which then acts to increase the transcription of genes necessary for entry into S phase. The catalytic activity of CDK4 is stimulated by binding to a partner protein, Cyclin D. One of the first demonstrations of a direct link between cancer and the cell cycle was made with the observation that the Cyclin D1 gene was amplified and cyclin D protein levels increased (and hence the activity of CDK4 increased) in many human tumours (Reviewed in Sherr, 1996, Science 274: 1672-1677; Pines, 1995, Seminars in Cancer Biology 6: 63-72). Other studies (Loda et al., 1997, Nature Medicine 3(2): 231-234; Gemma et al., 1996, International Journal of Cancer 68(5): 605-11; Elledge et al. 1996, Trends in Cell Biology 6; 388-392) have shown that negative regulators of CDK function are frequently down regulated or deleted in human tumours again leading to inappropriate activation of these kinases.

More recently, protein kinases that are structurally distinct from the CDK family have been identified which play critical roles in regulating the cell cycle and which also appear to be important in oncogenesis. They include the human homologues of the Drosophila aurora and S. cerevisiae Ipl1 proteins. The three human homologues of these genes aurora A, aurora B and aurora C (also known as aurora 2, aurora 1 and aurora 3 respectively) encode cell cycle regulated serine-threonine protein kinases (summarised in Adams et al., 2001, Trends in Cell Biology. 11(2): 49-54), which show a peak of expression and kinase activity through G2 and mitosis.

Several observations implicate the involvement of human aurora proteins in cancer. Firstly, it is known that the aurora A gene maps to chromosome 20q13, a region that is frequently amplified in human tumours including both breast and colon tumours. Aurora A may be the major target gene of this amplicon, since aurora A DNA is amplified and mRNA overexpressed in greater than 50% of primary human colorectal cancers. In these tumours aurora A protein levels appear greatly elevated compared to adjacent normal tissue. In addition, transfection of rodent fibroblasts with human aurora A leads to transformation, conferring the ability to grow in soft agar and form tumours in nude mice (Bischoff et al., 1998, The EMBO Journal. 17(11): 3052-3065). Other work (Zhou et al., 1998, Nature Genetics. 20(2): 189-93) has shown that artificial overexpression of aurora A leads to an increase in centrosome number and an increase in aneuploidy, a known event in the development of cancer.

It has also been shown that there is an increase in expression of aurora B (Adams et al., 2001, Chromsoma. 110(2):65-74) and aurora C (Kimura et al., 1999, Journal of Biological Chemistry, 274(11): 7334-40) in tumour cells when compared to normal cells. Aurora B is overexpressed in cancer cells and increased levels of Aurora B have been shown to correlate with advanced stages of colorectal cancer (Katayama et al (1999) J. Natl. Cancer Inst. 91:1160). Furthermore, one report suggests that overexpression of Aurora B induces aneuploidy through increased phosphorylation of histone H3 at serine 10 and that cells overexpressing Aurora B form more aggressive tumours that develop metastases (Ota, T. et al, 2002, Cancer Res. 62: 5168-5177). Aurora B is a chromosome passenger protein which exists in a stable complex with at least three other passenger proteins, Survivin, INCENP and Borealin (Carmena M. et al. 2003, Nat. Rev. Mol. Cell. Biol. 4: 842-854). Survivin is also upregulated in cancer and contains a BIR (Baculovirus Inhibitor of apoptosis protein (IAP) Repeat) domain and may therefore play a role in protecting tumour cells from apoptosis and/or mitotic catastrophe.

With regard to Aurora C, its expression is thought to be restricted to the testis but it has been found to be overexpressed in various cancer lines. (Katayarna H et al, 2003, Cancer and Metastasis Reviews 22: 451-464).

Importantly, it has been demonstrated that abrogation of aurora A expression and function by antisense oligonucleotide treatment of human tumour cell lines (WO 97/22702 and WO 99/37788) leads to cell cycle arrest and exerts an antiproliferative effect in these tumour cell lines. Additionally, small molecule inhibitors of aurora A and aurora B have been demonstrated to have an antiproliferative effect in human tumour cells (Keen et al. 2001, Poster #2455, American Association of Cancer research annual meeting), as has selective abrogation of aurora B expression alone by siRNA treatment (Ditchfield et al., 2003, Journal of Cell Biology, 161(2):267-280). This indicates that inhibition of the function of aurora A and/or aurora B will have an antiproliferative effect that may be useful in the treatment of human tumours and other hyperproliferative diseases. It is believed that inhibition of one or more aurora kinase as a therapeutic approach to such diseases may have significant advantages over targeting signalling pathways upstream of the cell cycle (e.g. those activated by growth factor receptor tyrosine kinases such as epidermal growth factor receptor (EGFR) or other receptors). As the cell cycle is ultimately downstream of all of these diverse signalling events, cell cycle directed therapies such as inhibition of one or more aurora kinase is predicted to be active across all proliferating tumour cells, whilst approaches directed at specific signalling molecules (e.g. EGFR) are believed to be active only in the subset of tumour cells which express those receptors. It is also believed that significant “cross talk” exists between these signalling pathways meaning that inhibition of one component may be compensated for by another.

A number of quinazoline derivatives have already been proposed for use in the inhibition of one or more aurora kinase. WO 02/00649 discloses quinazoline derivatives bearing a 5-membered heteroaromatic ring such as thiazole. WO 03/55491 discloses quinazoline derivatives substituted by a pyrazole ring. These compounds inhibit one or more aurora kinase and are able to inhibit the growth of cells from the human tumour cell line SW620. An example of such a compound is:

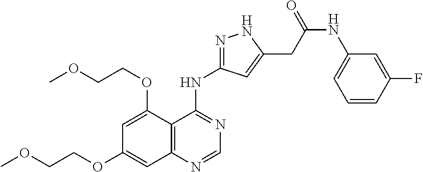

Further compounds having this activity are required but it would be advantageous if such compounds were additionally active in cells known to be resistant to chemotherapeutic agents and in particular in cells that over-express efflux proteins. Examples of efflux proteins include p-glycoprotein (MDR1), multidrug resistance associated proteins 1, 2, 3, 4 and 5, BCRP (MXR) and BSEP (SPGP) (Gottesman M. et al. 2001, Nature Reviews Cancer 2:48-58).

It would also be useful if such compounds had more advantageous physical properties that allowed them to be more effectively used in the treatment of hyperproliferative diseases such as cancer.

We have surprisingly found a small group of compounds, generally a selected subgroup of those described in WO 03/55491, which have superior activity in resistant cells and/or which have more advantageous physical properties. In particular the compounds of the invention display lower plasma protein binding and thus higher free drug levels in plasma. The compounds are therefore expected to be more efficacious. In addition it is believed that the compounds have better aqueous solubility.

The compounds particularly inhibit the effects of aurora A kinase and/or aurora B kinase and are therefore useful in the treatment of proliferative diseases such as cancer. In particular, the compounds may be used to treat solid or haematological tumours and more particularly any one of, or combination of, colorectal, breast, lung, prostate, bladder, renal or pancreatic cancer or leukaemia or lymphoma.

Accordingly, one aspect of the invention provides a compound of formula (I)

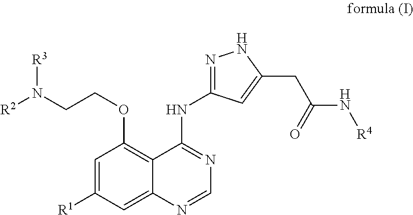

or a salt, ester or prodrug thereof;
wherein
R¹ is hydrogen or C₁₋₄alkoxy optionally substituted by C₁₋₄alkoxy;
R² is a group of formula (IA) wherein * is the point of attachment to formula (I);

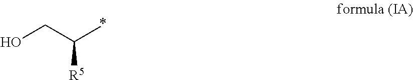

R³ is hydrogen or C₁₋₄alkyl optionally substituted by C₁₋₄alkoxy;
or R² and R³ together with the nitrogen atom to which they are attached form a ring of formula (IB) wherein * is the point of attachment to formula (I);

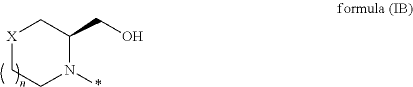

or R² and R³ together with the nitrogen atom to which they are attached form a ring of formula (IC) wherein * is the point of attachment to formula (I), provided that, in this case, R¹ is C₂₋₄alkoxy optionally substituted by C₁₋₄alkoxy;

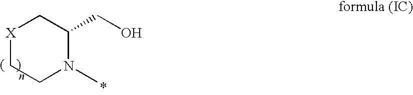

R⁴ is phenyl optionally substituted by 1 or 2 halo;
R⁵ is hydrogen or C₁₋₄alkyl optionally substituted by C₁₋₄alkoxy;
n is 0 or 1; and
X is CH₂, NH, N(C₁₋₄alkyl), O or S.

As a further aspect a compound of formula (J) or a pharmaceutically acceptable salt thereof is provided.

The invention also provides a compound of formula (I′)

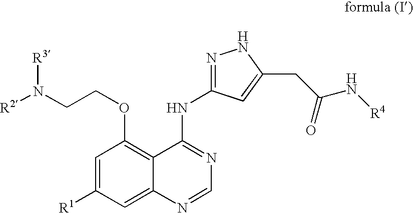

or a salt thereof;
wherein
R¹ is hydrogen or C₁₋₄alkoxy optionally substituted by C₁₋₄alkoxy;
R^{2′} is a group of formula (IA′) wherein * is the point of attachment to formula (I′);

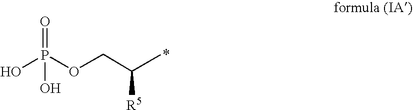

R^{3′} is hydrogen or C₁₋₄alkyl optionally substituted by C₁₋₄alkoxy;
or R^{2′} and R^{3′} together with the nitrogen atom to which they are attached form a ring of formula (IB′) wherein * is the point of attachment to formula (I′);

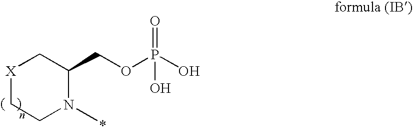

or R^{2′} and R^{3′} together with the nitrogen atom to which they are attached form a ring of formula (IC′) wherein * is the point of attachment to formula (I′), provided that in this case, R¹ is C₂₋₄alkoxy optionally substituted by C₁₋₄alkoxy;

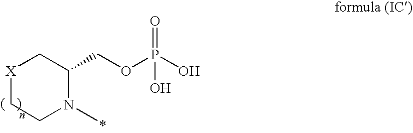

R⁴ is phenyl optionally substituted by 1 or 2 halo;
R⁵ is hydrogen or C₁₋₄alkyl optionally substituted by C₁₋₄alkoxy;
n is 0 or 1; and
X is CH₂, NH, N(C₁₋₄alkyl), O or S.

As a further aspect a compound of formula (I′) or a pharmaceutically acceptable salt thereof is provided.

In this specification the term alkyl when used either alone or as a suffix or prefix or otherwise includes straight-chain and branched-chain saturated structures comprising carbon and hydrogen atoms. References to individual alkyl groups such as propyl are specific for the straight-chain version only and references to individual branched-chain alkyl groups such as tert-butyl are specific for the branched chain version only.

The prefix Cₘ₋ₙ in Cₘ₋ₙalkyl and other terms (where m and n are integers) indicates the range of carbon atoms that are present in the group, for example C₁₋₄alkyl includes C₁alkyl (methyl), C₂alkyl (ethyl), C₃alkyl (propyl and isopropyl) and C₄alkyl (butyl, sec-butyl, isobutyl and tert-butyl).

The term Cₘ₋ₙalkoxy comprises —O—Cₘ₋ₙalkyl groups.

The term halo includes fluoro, chloro, bromo and iodo.

Phosphonooxy is a group of formula —OP(O)(OH)₂. However the term “phosphonooxy” may also include salts of this group such as those formed with alkali metal ions such as sodium or potassium ions or alkaline earth metal ions, for example calcium or magnesium ions.

This specification may make use of composite terms to describe groups comprising more than one functionality. Such terms are to be interpreted as is understood in the art.

Where optional substituents are chosen from 1 or 2 or more groups or substituents it is to be understood that this definition includes all substituents being chosen from one of the specified groups i.e. all substituents being the same, or the substituents being chosen from two or more of the specified groups i.e. the substituents not being the same.

Compounds of the present invention have been named with the aid of computer software (ACD/Name version 8.0).

Suitable values for any R group or any part or substituent for such groups include:

| | |
|---|---|
| for C₁₋₄alkyl: | methyl, ethyl, propyl, isopropyl, butyl, sec-butyl, |
| | isobutyl and tert-butyl; |
| for C₁₋₄alkoxy: | methoxy, ethoxy, propoxy, isopropoxy, butoxy, |
| | sec-butoxy, isobutoxy and tert-butoxy; |
| for C₂₋₄alkoxy: | ethoxy, propoxy, isopropoxy, butoxy, sec-butoxy, |
| | isobutoxy and tert-butoxy. |

Within the present invention it is to be understood that a compound of the invention may exhibit the phenomenon of tautomerism and that the formulae drawings within this specification can represent only one of the possible tautomeric forms. It is to be understood that the invention encompasses any tautomeric form which has aurora kinase inhibitory activity and in particular aurora A and/or aurora B kinase inhibitory activity and is not to be limited merely to any one tautomeric form utilized within the formulae drawings.

It is also to be understood that certain compounds of the invention and salts thereof can exist in solvated as well as unsolvated forms such as, for example, hydrated forms. It is to be understood that the invention encompasses all such solvated forms which have aurora kinase inhibitory activity and in particular aurora A and/or aurora B kinase inhibitory activity.

The present invention relates to the compounds of formula (I) or formula (I′) as herein defined as well as to the salts thereof. Salts for use in pharmaceutical compositions will be pharmaceutically acceptable salts, but other salts may be useful in the production of the compounds of formula (I) or formula (I′) and their pharmaceutically acceptable salts. Pharmaceutically acceptable salts of the invention may, for example, include acid addition salts of compounds of formula (I) or formula (I′) as herein defined which are sufficiently basic to form such salts. Such acid addition salts include but are not limited to fumarate, methanesulphonate, hydrochloride, hydrobromide, citrate and maleate salts and salts formed with phosphoric and sulphuric acid. In addition where compounds of formula (I) or formula (I′) are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

The compounds of formula (I) may also be provided as in vivo hydrolysable esters. An in vivo hydrolysable ester of a compound of formula (I) containing hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid. The compounds of formula (I′) are examples of in vivo hydrolysable esters of the compounds of formula (I).

Suitable pharmaceutically acceptable esters for hydroxy include inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example formyl, acetyl; benzoyl; phenylacetyl; substituted benzoyl and phenylacetyl; C₁₋₁₀alkoxycarbonyl (to give alkyl carbonate esters), for example ethoxycarbonyl; di-C₁₋₄alkylcarbamoyl and N-(di-C₁₋₄alkylaminoethyl)-N—C₁₋₄alkylcarbamoyl (to give carbamates); di-C₁₋₄alkylaminoacetyl and carboxyacetyl. Examples of ring substituents on phenylacetyl and benzoyl include aminomethyl, C₁₋₄alkylaminomethyl and di-(C₁₋₄alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)OC₁₋₆alkyl-CO—, wherein R^{A} is for example, benzyloxy-C₁₋₄alkyl or phenyl. Suitable substituents on a phenyl group in such esters include, for example, 4-C₁₋₄piperazino-C₁₋₄alkyl, piperazino-C₁₋₄alkyl and morpholino-C₁₋₄alkyl.

The compounds of the formula (I) may be also be administered in the form of a prodrug which is broken down in the human or animal body to give a compound of the formula (I). Examples of prodrugs include in vivo hydrolysable esters of a compound of the formula (I). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);

### c) H. Bundgaard, Advanced Drug Delivery Reviews, 8, 1-38 (1992);

### d) H. Bundgaard, et al., Journal of Pharmaceutical Sciences, 77, 285 (1988); and

### e) N. Kakeya, et al., Chem Pharm Bull, 32, 692 (1984).

Particular values of R¹, R⁴, R⁵, n and X for compounds of formula (I) and/or formula (I′) are as follows. Such values may be used where appropriate with any of the definitions, claims or embodiments defined herein.

In one aspect of the invention R¹ is C₁₋₄alkoxy optionally substituted by methoxy. In another aspect R¹ is methoxy, ethoxy or methoxyethoxy.

In one aspect of the invention R⁴ is phenyl optionally substituted by 1 or 2 fluoro or chloro. In another aspect R⁴ is phenyl, 3-fluorophenyl, 2,3-difluorophenyl, 2,4-difluorophenyl, 2,5-difluorophenyl, 3,5-difluorophenyl, 2-fluoro-3-chlorophenyl or 2-fluoro-4-chlorophenyl. In a further aspect R⁴ is 3-fluorophenyl or 2,3-difluorophenyl.

In one aspect of the invention R⁵ is hydrogen, methyl, ethyl or isopropyl. In another aspect R⁵ is hydrogen or methyl.

In one aspect of the invention n is 0.

In one aspect of the invention X is CH₂, NH or NMe. In another aspect X is CH₂.

Particular values of R² and R³ for a compound of formula (I) are as follows. Such values may be used where appropriate with any of the definitions, claims or embodiments defined herein.

In one aspect of the invention R² is 2-hydroxyethyl, (1S)-2-hydroxy-1-methylethyl, (1S)-2-hydroxy-1-ethylethyl, (1S)-2-hydroxy-1-isopropyl ethyl or (1S)-2-hydroxy-1-(methoxymethyl)ethyl. In another aspect R² is 2-hydroxyethyl or (1S)-2-hydroxy-1-methylethyl.

In one aspect of the invention R³ is hydrogen, methyl, ethyl or methoxyethyl.

In one aspect of the invention R² and R³ together with the nitrogen atom to which they are attached form:

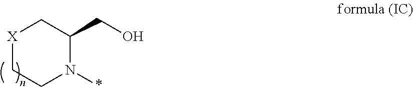

In another aspect R² and R³ together with the nitrogen atom to which they are attached form:

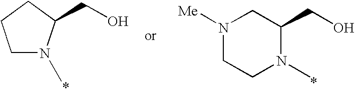

where * is the point of attachment to formula (I); or R² and R³ together with the nitrogen atom to which they are attached form:

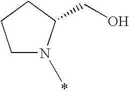

where * is the point of attachment to formula (I) and provided that, in this case, R¹ is ethoxy or methoxyethoxy.

In another aspect R² and R³ together with the nitrogen atom to which they are attached form:

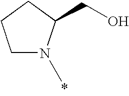

where * is the point of attachment to formula (I); or R² and R³ together with the nitrogen atom to which they are attached form:

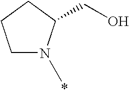

where * is the point of attachment to formula (I) and provided that, in this case, R¹ is ethoxy or methoxyethoxy.

In a further aspect R² and R³ together with the nitrogen atom to which they are attached form:

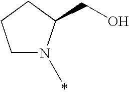

where * is the point of attachment to formula (I).

Particular values of R^{2′} and R^{3′} for a compound of formula (I′) are as follows. Such values may be used where appropriate with any of the definitions, claims or embodiments defined herein.

In one aspect of the invention R^{2′} is 2-phosphonooxyethyl, (1S)-2-phosphonooxy-1-methylethyl, (1S)-2-phosphonooxy-1-ethylethyl, (1S)-2-phosphonooxy-1-isopropylethyl or (1S)-2-phosphonooxy-1-(methoxymethyl)ethyl. In another aspect R² is 2-phosphonooxyethyl or (1S)-2-phosphonooxy-1-methylethyl.

In one aspect of the invention R^{3′} is hydrogen, methyl, ethyl or methoxyethyl.

In one aspect of the invention R^{2′} and R^{3′} together with the nitrogen atom to which they are attached form:

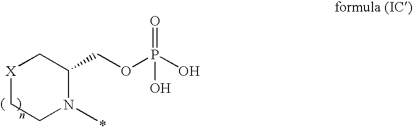

In another aspect R^{2′} and R^{3′} together with the nitrogen atom to which they are attached form:

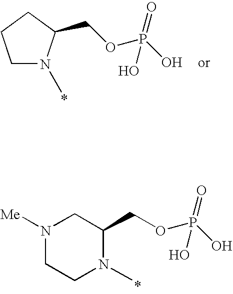

where * is the point of attachment to formula (I); or R^{2′} and R^{3′} together with the nitrogen atom to which they are attached form:

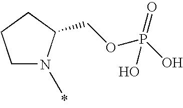

where * is the point of attachment to formula (I) and provided that in this case R¹ is ethoxy or methoxyethoxy.

In another aspect R^{2′} and R^{3′} together with the nitrogen atom to which they are attached form:

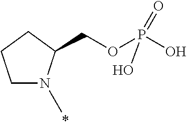

where * is the point of attachment to formula (I); or R^{2′} and R^{3′} together with the nitrogen atom to which they are attached form:

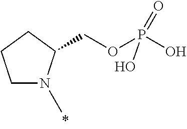

where * is the point of attachment to formula (I) and provided that, in this case, R¹ is ethoxy or methoxyethoxy.

In a further aspect R^{2′} and R^{3′} together with the nitrogen atom to which they are attached form:

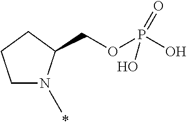

where * is the point of attachment to formula (I).

A particular class of compounds is of formula (I) wherein:

R¹ is C₁₋₄alkoxy optionally substituted by methoxy;
R² is a group of formula (IA) wherein * is the point of attachment to formula (I);

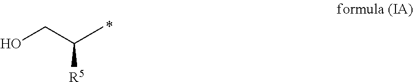

R³ is hydrogen or C₁₋₄alkyl optionally substituted by C₁₋₄alkoxy;
or R² and R³ together with the nitrogen atom to which they are attached form a ring of formula (IB) wherein * is the point of attachment to formula (I);

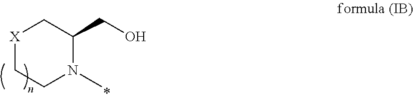

or R² and R³ together with the nitrogen atom to which they are attached form a ring of formula (IC) wherein * is the point of attachment to formula (I), provided that, in this case, R¹ is C₂₋₄alkoxy optionally substituted by methoxy;

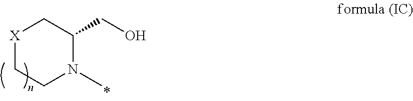

R⁴ is phenyl optionally substituted by 1 or 2 fluoro or chloro;
R⁵ is hydrogen, methyl, ethyl or isopropyl;
n is 0 or 1; and
X is CH₂, NH or NMe; or a salt, ester or prodrug thereof.

A particular class of compounds is of formula (I) wherein:

R¹ is methoxy, ethoxy or methoxyethoxy;
R² is 2-hydroxyethyl, (1S)-2-hydroxy-1-methylethyl, (1S)-2-hydroxy-1-ethylethyl, (1S)-2-hydroxy-1-isopropylethyl or (1S)-2-hydroxy-1-(methoxymethyl)ethyl;
R³ is hydrogen, methyl, ethyl or methoxyethyl; or R² and R³ together with the nitrogen atom to which they are attached form:

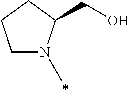

where * is the point of attachment to formula (I); or R² and R³ together with the nitrogen atom to which they are attached form:

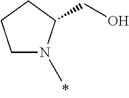

where * is the point of attachment to formula (I) and provided that R¹ is ethoxy or methoxyethoxy; and
R⁴ is phenyl, 3-fluorophenyl, 2,3-difluorophenyl, 2,4-difluorophenyl, 2,5-difluorophenyl, 2-fluoro-3-chlorophenyl or 2-fluoro-4-chlorophenyl;
or a salt, ester or prodrug thereof.

Another particular class of compounds is of formula (I) wherein:

R¹ is methoxy, ethoxy or methoxyethoxy;
R² is 2-hydroxyethyl or (1S)-2-hydroxy-1-methylethyl;
R³ is hydrogen, methyl, ethyl or methoxyethyl; or
R² and R³ together with the nitrogen atom to which they are attached form:

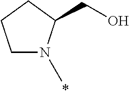

where * is the point of attachment to formula (I); and
R⁴ is 3-fluorophenyl or 2,3-difluorophenyl;
or a salt, ester or prodrug thereof.

A particular class of compounds is of formula (I′) wherein:

R¹ is C₁₋₄alkoxy optionally substituted by methoxy;
R^{2′} is a group of formula (IA) wherein * is the point of attachment to formula (I);

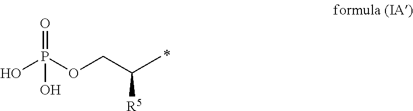

R^{3′} is hydrogen or C₁₋₄alkyl optionally substituted by C₁₋₄alkoxy;
or R^{2′} and R^{3′} together with the nitrogen atom to which they are attached form a ring of formula (IB) wherein * is the point of attachment to formula (I);

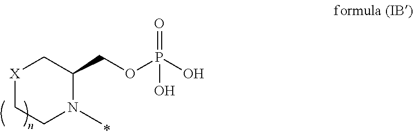

or R^{2′} and R^{3′} together with the nitrogen atom to which they are attached form a ring of formula (IC′) wherein * is the point of attachment to formula (I′)), provided that, in this case, R¹ is C₂₋₄alkoxy optionally substituted by methoxy;

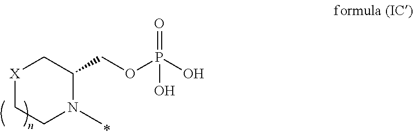

R⁴ is phenyl optionally substituted by 1 or 2 fluoro or chloro;
R⁵ is hydrogen, methyl, ethyl or isopropyl;
n is 0 or 1; and

### X is CH₂, NH or NMe;

or a salt thereof.

A particular class of compounds is of formula (I′) wherein:

R¹ is methoxy, ethoxy or methoxyethoxy;
R^{2′} is 2-phosphonooxyethyl or (1S)-2-phosphonooxy-1-methylethyl;
R^{3′} is hydrogen, methyl, ethyl or methoxyethyl;
or R^{2′} and R^{3′} together with the nitrogen atom to which they are attached form:

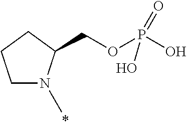

where * is the point of attachment to formula (I);
R⁴ is 3-fluorophenyl or 2,3-difluorophenyl;
or a salt thereof.

Particular embodiments of the invention are any one of, or any combination of the following compounds:
N-(3-fluorophenyl)-2-{3-[(5-{2-[(2-hydroxyethyl)(methyl)amino]ethoxy}-7-methoxyquinazolin-4-yl)amino]-1H-pyrazol-5-yl}acetamide;
N-(3-fluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(3-fluorophenyl)-2-{3-[(5-{2-[(2S)-2-(hydroxymethyl)pyrrolidin-1-yl]ethoxy}-7-methoxyquinazolin-4-yl)amino]-1H-pyrazol-5-yl}acetamide;
N-(3-fluorophenyl)-2-(3-{[5-(2-{[(1S)-2-hydroxy-1-methylethyl]amino}ethoxy)-7-methoxyquinazolin-4-yl]amino}-1H-pyrazol-5-yl)acetamide;
N-(2,3-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(2,3-difluorophenyl)-2-{3-[(5-{2-[ethyl(2-hydroxyethyl)amino]ethoxy}-7-methoxyquinazolin-4-yl)amino]-1H-pyrazol-5-yl}acetamide;
N-(2,3-difluorophenyl)-2-{3-[(5-{2-[(2S)-2-(hydroxymethyl)pyrrolidin-1-yl]ethoxy}-7-methoxyquinazolin-4-yl)amino]-1H-pyrazol-5-yl}acetamide;
N-(2,3-difluorophenyl)-2-{3-[(5-{2-[(2S)-2-(hydroxymethyl)-4-methylpiperazin-1-yl]ethoxy}-7-methoxyquinazolin-4-yl)amino]-1H-pyrazol-5-yl}acetamide;
N-(2,3-difluorophenyl)-2-{3-[(5-{2-[(2-hydroxyethyl)(methyl)amino]ethoxy}-7-methoxyquinazolin-4-yl)amino]-1H-pyrazol-5-yl}acetamide;
N-(2,3-difluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide;
N-(2,3-difluorophenyl)-2-(3-{[5-{2-[ethyl(2-hydroxyethyl)amino]ethoxy}-7-(2-methoxyethoxy)quinazolin-4-yl]amino}-1H-pyrazol-5-yl)acetamide;
N-(2,3-difluorophenyl)-2-(3-{[5-{2-[(2S)-2-(hydroxymethyl)pyrrolidin-1-yl]ethoxy}-7-(2-methoxyethoxy)quinazolin-4-yl]amino}-1H-pyrazol-5-yl)acetamide;
N-(2,3-difluorophenyl)-2-{3-[(7-ethoxy-5-{2-[(2-hydroxyethyl)(methyl)amino]ethoxy}quinazolin-4-yl)amino]-1H-pyrazol-5-yl}acetamide;
N-(2,3-difluorophenyl)-2-{3-[(7-ethoxy-5-{2-[(2S)-2-(hydroxymethyl)pyrrolidin-1-yl]ethoxy}quinazolin-4-yl)amino]-1H-pyrazol-5-yl}acetamide; and
N-(2,3-difluorophenyl)-2-{3-[(7-ethoxy-5-{2-[(2-hydroxyethyl)(2-methoxyethyl)amino]ethoxy}quinazolin-4-yl)amino]-1H-pyrazol-5-yl}acetamide;
   or a salt, ester or prodrug thereof and more particularly a pharmaceutically acceptable salt thereof.

Further particular embodiments of the invention are any one of, or any combination of, the following compounds:
N-(3-fluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide;
N-(2,3-difluorophenyl)-2-(3-{[7-ethoxy-5-(2-{[(1S)-2-hydroxy-1-methylethyl]amino}ethoxy)quinazolin-4-yl]amino}-1H-pyrazol-5-yl)acetamide;
N-(2,3-difluorophenyl)-2-(3-{[7-ethoxy-5-(2-{[(1S)-1-(hydroxymethyl)propyl]amino}ethoxy)quinazolin-4-yl]amino}-1H-pyrazol-5-yl)acetamide;
N-(2,3-difluorophenyl)-2-(3-{[7-ethoxy-5-(2-{[(1S)-1-(hydroxymethyl)-2-methylpropyl]amino}ethoxy)quinazolin-4-yl]amino}-1H-pyrazol-5-yl)acetamide;
N-(2,3-difluorophenyl)-2-(3-{[7-ethoxy-5-(2-{[(1R)-2-hydroxy-1-(methoxymethyl)ethyl]amino}ethoxy)quinazolin-4-yl]amino}-1H-pyrazol-5-yl)acetamide;
2-{3-[(5-{2-[(2-hydroxyethyl)(2-methoxyethyl)amino]ethoxy}-7-methoxyquinazolin-4-yl)amino]-1H-pyrazol-5-yl}-N-phenylacetamide;
N-(2,4-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(3,5-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(2,5-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(2,3-difluorophenyl)-2-(3-{[5-{2-[(2R)-2-(hydroxymethyl)pyrrolidin-1-yl]ethoxy}-7-(2-methoxyethoxy)quinazolin-4-yl]amino}-1H-pyrazol-5-yl)acetamide;
N-(4-chloro-2-fluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide; or
N-(3-chloro-2-fluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide;
   or a salt, ester or prodrug thereof and more particularly a pharmaceutically acceptable salt thereof.

Further embodiments of the invention are any one of, or any combination of, the following compounds:
2-[(2-{[4-[(5-{2-[(2,3-difluorophenyl)amino]-2-oxoethyl}-1H-pyrazol-3-yl)amino]-7-(2-methoxyethoxy)quinazolin-5-yl]oxy}ethyl)(methyl)amino]ethyl dihydrogen phosphate;
2-[(2-{[4-[(5-{2-[(2,3-difluorophenyl)amino]-2-oxoethyl}-1H-pyrazol-3-yl)amino]-7-(2-methoxyethoxy)quinazolin-5-yl]oxy}ethyl)(ethyl)amino]ethyl dihydrogen phosphate;
[(2S)-1-(2-{[4-[(5-{2-[(2,3-difluorophenyl)amino]-2-oxoethyl}-1H-pyrazol-3-yl)amino]-7-(2-methoxyethoxy)quinazolin-5-yl]oxy}ethyl)pyrrolidin-2-yl]methyl dihydrogen phosphate;
2-[[2-({4-[(5-{2-[(2,3-difluorophenyl)amino]-2-oxoethyl}-1H-pyrazol-3-yl)amino]-7-methoxyquinazolin-5-yl}oxy)ethyl](2-methoxyethyl)amino]ethyl dihydrogen phosphate;
{(2S)-1-[2-({4-[(5-{2-[(2,3-difluorophenyl)amino]-2-oxoethyl}-1H-pyrazol-3-yl)amino]-7-methoxyquinazolin-5-yl}oxy)ethyl]pyrrolidin-2-yl}methyl dihydrogen phosphate;
2-[[2-({4-[(5-{2-[(2,3-difluorophenyl)amino]-2-oxoethyl}-1H-pyrazol-3-yl)amino]-7-methoxyquinazolin-5-yl}oxy)ethyl](methyl)amino]ethyl dihydrogen phosphate; and
(2S)-2-{[2-({4-[(5-{2-[(3-fluorophenyl)amino]-2-oxoethyl}-1H-pyrazol-3-yl)amino]-7-methoxyquinazolin-5-yl}oxy)ethyl]amino}propyl dihydrogen phosphate;
   or a salt thereof and more particularly a pharmaceutically acceptable salt thereof.

The present invention also provides a process for the preparation of a compound of formula (I) or a salt, ester or prodrug thereof, which process comprises reacting a compound of formula (II) where L¹ is a leaving group such as halo (e.g. chloro):

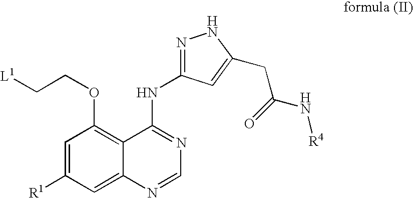

with an amine of formula (III):

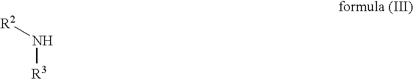

where R¹, R², R³ and R⁴ are as defined herein and thereafter if necessary:
i) converting a compound of formula (I) into another compound of formula (I);
ii) removing any protecting groups; and/or
iii) forming a salt, ester or prodrug.
Suitable reaction conditions for this process include heating a compound of formula (II) with an excess of amine of formula (III) in an inert solvent such as dimethylacetamide, with or without the addition of a suitable catalyst (such as tetra-n-butylammoniuim iodide or potassium iodide) at a temperature of 50 to 100° C. for 12 to 72 hours. Alternatively, the leaving group L¹ in formula (II) may be a carboxaldehyde in which case the reaction with amine (III) may be carried out under reductive conditions using a reducing agent such as sodium cyanoborohydride.

The amines of formula (III) are known in the art or may be prepared by the skilled person using methods known in the art.

The process may further comprise a method for the preparation of a compound of formula (II) which method comprises the reaction of a compound of formula (IV) where L² is a leaving group such as halo (e.g. chloro):

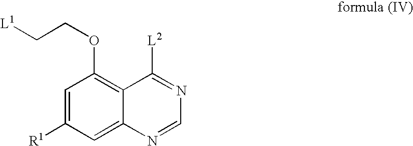

with a compound of formula (V):

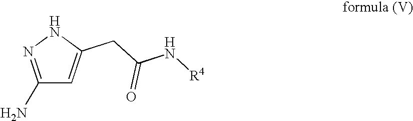

This reaction may be performed under a range of conditions described in the literature such as heating a compound of formula (IV) with a compound of formula (V) in a solvent such as isopropanol or dimethylacetamide, in the presence or absence of an acid catalyst such as hydrochloric acid, at a temperature of 20 to 100° C. for 2 to 24 hours.

The process may alternatively comprise a method for the preparation of a compound of formula (II) which method comprises the reaction of a compound of formula (VI):

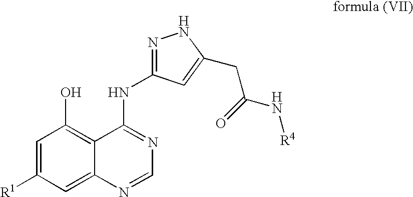

with a compound of formula (III):

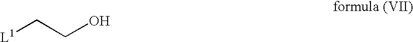

wherein L¹ is as described herein.
This reaction can be performed under a range of conditions described in the literature such as coupling a compound of formula (VI) with a compound of formula (VII) in a solvent such as tetrahydrofuran, in the presence of a suitable coupling reagent such as di-tert-butylazodicarboxylate and a suitable phosphine such as triphenylphosphine, at a temperature of 20 to 60° C. for 1 to 5 hours.

The process may alternatively comprise a method for the preparation of a compound of formula (II) which method comprises the reaction of a compound of formula (VIII):

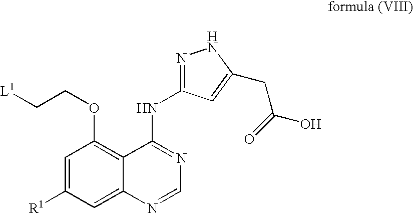

wherein L¹ is as defined herein; with a compound of formula (IX)

R⁴—NH₂ formula (IX)

This reaction can be performed under a range of conditions described in the literature such as coupling a compound of formula (VIII) with a compound of formula (IX) in a solvent such as dimethylformamide, in the presence of a suitable coupling reagent such as pentafluorophenyltrifluoroacetate and pyridine, at a temperature of 0 to 60° C. for 1 to 24 hours.

Compounds of formula (IV) can be prepared from a compound of formula (X):

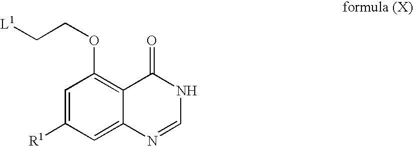

with a suitable chlorinating agent such as phosphorus oxychloride in a suitable solvent such as 1,2-dichloroethane or acetonitrile in the presence of a suitable base such as di-iso-propylethyl amine, at a temperature of 0 to 80° C. for 2 to 24 hours.

Compounds of formula (X) are either known compounds or they can be prepared by conventional methods. In particular, compounds of formula (X) may be prepared, by reaction of a compound of formula (XI):

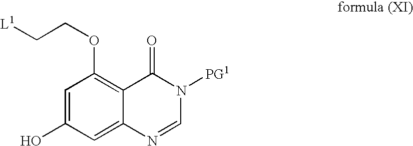

where PG¹ is a suitable protecting group such as pivaloyloxymethyl, with a compound of formula (XII):

R¹—OH formula (XII)

where R¹ is as defined herein; followed by removal of protecting group PG¹.
This reaction can be performed under a range of conditions described in the literature such as coupling a compound of formula (XI) with a compound of formula (XII) in a solvent such as tetrahydrofuran, in the presence of a suitable coupling reagent such as di-tert-butylazodicarboxylate and a suitable phosphine such as triphenylphosphine, at a temperature of 20 to 60° C. for 1 to 5 hours.

Compounds of formula (XI) are either known compounds or they can be prepared by conventional methods. In particular, compounds of formula (XI) may be prepared by reaction of a compound of formula (XIII):

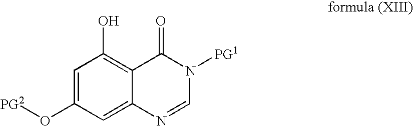

where PG¹ and PG² are suitable protecting group such as pivaloyloxymethyl and benzyl respectively; with a compound of formula (VII) in a solvent such as tetrahydrofuran, in the presence of a suitable coupling reagent such as di-tert-butylazodicarboxylate and a suitable phosphine such as triphenylphosphine, at a temperature of 20 to 60° C. for 1 to 5 hours; followed by removal of protecting group PG².

Compounds of formula (XIII) are made according to the literature and in particular the compound where PG¹ is pivaloyloxymethyl and PG² is benzyl is known in the literature.

The process may further comprise a method for the preparation of a compound of formula (VI) which method comprises the reaction of a compound of formula (XIV):

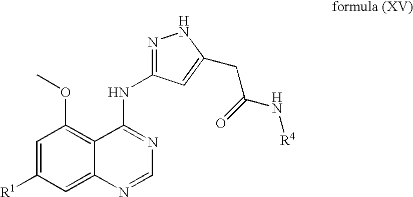

with a suitable de-methylating reagent, such as pyridine hydrochloride, in a suitable solvent such as pyridine, at a temperature of 90 to 110° C. for 5 to 8 hours.

Compounds of formula (XIV) can be prepared by a reaction of a compound of formula (XV):

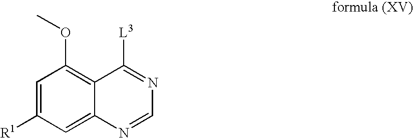

where L³ is a leaving group such as halo (e.g. chloro), with a compound of formula (V). This reaction can be performed under a range of conditions described in the literature such as heating a compound of formula (XV) with a compound of formula (V) in a solvent such as isopropanol or dimethylacetamide, in the presence or absence of an acid catalyst such as hydrochloric acid, at a temperature of 20 to 100° C. for 2 to 24 hours.

Compounds of formula (XV) are either known compounds or they can be prepared by conventional methods. In particular, compounds of formula (XV) may be prepared by reaction of a compound of formula (XVI):

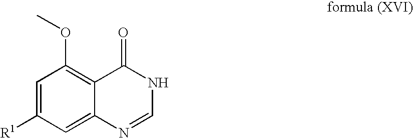

with a suitable chlorinating agent such as phosphorus oxychloride in a suitable solvent such as 1,2-dichloroethane or acetonitrile in the presence of a suitable base such as di-iso-propylethyl amine, at a temperature of 0 to 80° C. for 2 to 24 hours.

Compounds of formula (XVI) are either known compounds or they can be prepared by conventional methods. In particular, compounds of formula (XVI) may be prepared by reaction of a compound of formula (XVII):

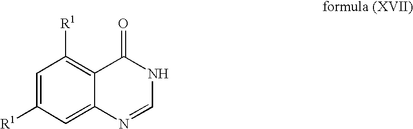

with sodium methoxide in a suitable solvent such as dimethylformamide at a temperature such as 110° C. for 12 to 24 hours.

Compounds of formula (XVII) are either known compounds or they can be prepared by conventional methods. In particular, compounds of formula (XV) may be prepared by reaction of a compound of formula (XII), where R¹ is as defined herein, with 5,7-difluoroquinazolone in a solvent such as dimethylformamide at a temperature such as 90° C. for 2 to 12 hours.

The process may further comprise a method for the preparation of a compound of formula (V) which method comprises the reaction of a compound of formula (XVIII):

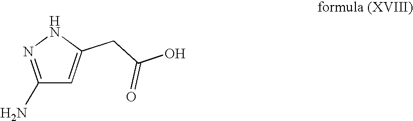

with a compound of formula (IX) in the presence of a coupling reagent, such as pentafluorophenyl trifluoroacetate and pyridine in a solvent such as dimethylformamide under inert and anhydrous conditions.

Compounds of formula (XVIII) are made according to the literature.

The process may further comprise a method for the preparation of a compound of formula (VIII) which method comprises the reaction of a compound of formula (IV) with a compound of formula (XVIII). This reaction can be performed under a range of conditions described in the literature such as coupling a compound of formula (IV) with a compound of formula (XVIII) in a solvent such as isopropanol or dimethylacetamide, in the presence or absence of an acid catalyst such as hydrochloric acid, at a temperature of 20 to 100° C. for 2 to 24 hours.

In another aspect the present invention provides a process for the preparation of a compound of formula (I′) or a salt thereof, which process comprises converting a compound of formula (I) into a compound of formula (I′) by phosphorylation of an appropriate hydroxy group and thereafter if necessary:

i) converting a compound of the formula (I′) into another compound of the formula (I′);
ii) removing any protecting groups; and/or
iii) forming a salt.

Phosphorylation may be suitably performed by treatment with 1-H tetrazole (or a suitable replacement such as S-ethyl tetrazole or pyridinium hydrochloride) and di-tert-butyldiethylphosphoramidite at 5 to 35° C. under an inert atmosphere for 30 minutes to 4 hours followed by treatment with an oxidizing agent such as meta-chloroperbenzoic acid (mCPBA) or 30% aqueous hydrogen peroxide at −10 to 25° C. for 2 to 18 hour. Deprotection of the tert-butyl groups to yield the phosphate group is required as a final step with these reagents and may be readily achieved by treatment with 4.0 N hydrochloric acid in 1,4-dioxane at 10 to 35° C. for 12 to 18 hours.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

According to a further aspect of the invention there is provided a pharmaceutical composition which comprises a compound of formula (I), or a pharmaceutically acceptable salt, ester or prodrug thereof, as defined herein in association with a pharmaceutically acceptable diluent or carrier.

Also provided is a pharmaceutical composition which comprises a compound of formula (I′), or a pharmaceutically acceptable salt thereof, as defined herein in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, soya bean oil, coconut oil, or preferably olive oil, or any other acceptable vehicle.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible or lyophilised powders and granules suitable for preparation of an aqueous suspension or solution by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, solutions, emulsions or particular systems, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in polyethylene glycol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30 μm or much less preferably 5 μm or less and more preferably between 5 μm and 1 μm, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

Therefore in a further aspect of the invention there is provided a compound of formula (I), or a pharmaceutically acceptable salt, ester or prodrug thereof, for use in therapy. In addition a compound of formula (I′) or a pharmaceutically acceptable salt thereof is provided for use in therapy.

Further provided is a compound of formula (I), or a pharmaceutically acceptable salt, ester or prodrug thereof, for use as a medicament and also provided is a compound of formula (I′), or a pharmaceutically acceptable salt thereof, for use as a medicament. Another aspect of the invention provides a compound of formula (I), or a pharmaceutically acceptable salt, ester or prodrug thereof, for use as a medicament for the treatment of hyperproliferative diseases such as cancer and in particular for the treatment of any one of, or any combination of, colorectal, breast, lung, prostate, bladder, renal or pancreatic cancer or leukaemia or lymphoma. Also provided is a compound of formula (I′), or a pharmaceutically acceptable salt thereof, for use as a medicament for the treatment of hyperproliferative diseases such as cancer and in particular for the treatment of any one of, or any combination of, colorectal, breast, lung, prostate, bladder, renal or pancreatic cancer or leukaemia or lymphoma.

Additionally a compound of formula (I), or a pharmaceutically acceptable salt, ester or prodrug thereof is provided for use in a method of treatment of a warm-blooded animal such as man by therapy. A compound of formula (I′) or a pharmaceutically acceptable salt thereof is also provided for use in a method of treatment of a warm-blooded animal such as man by therapy. Another aspect of the invention provides a compound of formula (I), or a pharmaceutically acceptable salt, ester or prodrug thereof, for use in a method of treatment of hyperproliferative diseases such as cancer and in particular treatment of any one of, or of any combination of, colorectal, breast, lung, prostate, bladder, renal or pancreatic cancer or leukaemia or lymphoma. Also provided is a compound of formula (I′), or a pharmaceutically acceptable salt thereof, for use in a method of treatment of hyperproliferative diseases such as cancer and in particular treatment of any one of, or of any combination of, colorectal, breast, lung, prostate, bladder, renal or pancreatic cancer or leukaemia or lymphoma.

In another aspect of the invention, there is provided the use of a compound of formula (I) or a pharmaceutically acceptable salt, ester or prodrug thereof, in the preparation of a medicament for the treatment of a disease where the inhibition of one or more aurora kinase(s) is beneficial. The use of a compound of formula (I′) or a pharmaceutically acceptable salt thereof in the preparation of a medicament for the treatment of a disease where the inhibition of one or more aurora kinase(s) is beneficial is also provided. In particular it is envisaged that inhibition of aurora A kinase and/or aurora B kinase may be beneficial. Preferably inhibition of aurora B kinase is beneficial. In another aspect of the invention, there is provided the use of a compound of formula (I) or a pharmaceutically acceptable salt, ester or prodrug thereof, in the preparation of a medicament for the treatment of hyperproliferative diseases such as cancer and in particular for the treatment of any one of, or for any combination of, colorectal, breast, lung, prostate, bladder, renal or pancreatic cancer or leukaemia or lymphoma. Also provided is the use of a compound of formula (I′) or a pharmaceutically acceptable salt thereof in the preparation of a medicament for the treatment of hyperproliferative diseases such as cancer and in particular for the treatment of any one of, or for any combination of, colorectal, breast, lung, prostate, bladder, renal or pancreatic cancer or leukaemia or lymphoma.

According to yet another aspect, there is provided a compound of formula (I) or a pharmaceutically acceptable salt, ester or prodrug thereof for use in a method of treating a human suffering from a disease in which the inhibition of one or more aurora kinase is beneficial, comprising the steps of administering to a person in need thereof a therapeutically effective amount of a compound of formula (I) or a pharmaceutically acceptable salt, ester or prodrug thereof. Further provided is a compound of formula (I′) or a pharmaceutically acceptable salt thereof for use in a method of treating a human suffering from a disease in which the inhibition of one or more aurora kinases is beneficial, comprising the steps of administering to a person in need thereof a therapeutically effective amount of a compound of formula (I′) or a pharmaceutically acceptable salt thereof. In particular it is envisaged that inhibition of aurora A kinase and/or aurora B kinase may be beneficial. Preferably inhibition of aurora B kinase is beneficial. Further provided is a compound of formula (I) or a pharmaceutically acceptable salt, ester or prodrug thereof for use in a method of treating a to human suffering from a hyperproliferative disease such as cancer and in particular from any one of, or from any combination of, colorectal, breast, lung, prostate, bladder, renal or pancreatic cancer or leukaemia or lymphoma, comprising the steps of administering to a person in need thereof a therapeutically effective amount of a compound of formula (I) or a pharmaceutically acceptable salt, ester or prodrug thereof. A compound of formula (I′) is also provided for use in a method of treating a human suffering from a hyperproliferative disease such as cancer and in particular from any one of, or from any combination of, colorectal, breast, lung, prostate, bladder, renal or pancreatic cancer or leukaemia or lymphoma, comprising the steps of administering to a person in need thereof a therapeutically effective amount of a compound of formula (I′) or a pharmaceutically acceptable salt thereof. The use of a compound of formula (I) or a pharmaceutically acceptable salt, ester or prodrug thereof in any of the methods of treating a human described above also form aspects of this invention. Additionally the use of a compound of formula (I′) or a pharmaceutically acceptable salt thereof in any of the methods of treating a human described above form other aspects of this invention.

For the above mentioned therapeutic uses the dose administered will vary with the compound employed, the mode of administration, the treatment desired, the disorder indicated e and the age and sex of the animal or patient. The size of the dose would thus be calculated according to well known principles of medicine.

In using a compound of formula (I) or formula (I′) for therapeutic or prophylactic purposes it will generally be administered so that a daily dose in the range, for example, 0.05 mg/kg to 50 mg/kg body weight is received, given if required in divided doses. In general lower doses will be administered when a parenteral route is employed. Thus, for example, for intravenous administration, a dose in the range, for example, 0.05 mg/kg to 25 mg/kg body weight (and in particular 0.05 mg/kg to 15 mg/kg body weight) will generally be used. Similarly, for administration by inhalation, a dose in the range, for example, 0.05 mg/kg to 25 mg/kg body weight (and in particular 0.05 mg/kg to 15 mg/kg body weight) will be used.

The treatment defined herein may be applied as a sole therapy or may involve, in addition to the compound of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestratrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine-threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

In addition a compound of the invention or a pharmaceutically acceptable salt, ester or prodrug thereof, may be used in combination with one or more cell cycle inhibitors. In particular with cell cycle inhibitors which inhibit bub1, bubR1 or CDK.

Such conjoint treatment may be achieved by way of the simultaneous, sequential or separate dosing of the individual components of the treatment. Such combination products employ the compounds of this invention within the dosage range described herein and the other pharmaceutically-active agent within its approved dosage range.

In addition to their use in therapeutic medicine, a compound of formula (I) and a pharmaceutically acceptable salt, ester or prodrug thereof are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

The compounds of the invention inhibit the serine-threonine kinase activity of the aurora kinases, in particular aurora A kinase and/or aurora B kinase and thus inhibit the cell cycle and cell proliferation. Compounds which inhibit aurora B kinase are of particular interest. The compounds are also active in resistant cells and have advantageous physical properties. These properties may be assessed for example, using one or more of the procedures set out below.

### (a) In Vitro Aurora A Kinase Inhibition Test

This assay determines the ability of a test compound to inhibit serine-threonine kinase activity. DNA encoding aurora A may be obtained by total gene synthesis or by cloning. This DNA may then be expressed in a suitable expression system to obtain polypeptide with serine-threonine kinase activity. In the case of aurora A, the coding sequence was isolated from cDNA by polymerase chain reaction (PCR) and cloned into the BamH1 and Not1 restriction endonuclease sites of the baculovirus expression vector pFastBac HTc (GibcoBRL/Life technologies). The 5′ PCR primer contained a recognition sequence for the restriction endonuclease BamH1 5′ to the aurora A coding sequence. This allowed the insertion of the aurora A gene in frame with the 6 histidine residues, spacer region and rTEV protease cleavage site encoded by the pFastBac HTc vector. The 3′ PCR primer replaced the aurora A stop codon with additional coding sequence followed by a stop codon and a recognition sequence for the restriction endonuclease NotI. This additional coding sequence (5′ TAC CCA TAC GAT GTT CCA GAT TAC GCT TCT TAA 3′) encoded for the polypeptide sequence YPYDVPDYAS. This sequence, derived from the influenza hemagglutin protein, is frequently used as a tag epitope sequence that can be identified using specific monoclonal antibodies. The recombinant pFastBac vector therefore encoded for an N-terminally 6 his tagged, C terminally influenza hemagglutin epitope tagged Aurora-A protein. Details of the methods for the assembly of recombinant DNA molecules can be found in standard texts, for example Sambrook et al. 1989, Molecular Cloning—A Laboratory Manual, 2^{nd} Edition, Cold Spring Harbor Laboratory press and Ausubel et al. 1999, Current Protocols in Molecular Biology, John Wiley and Sons Inc.

Production of recombinant virus can be performed following manufacturer's protocol from GibcoBRL. Briefly, the pFastBac-1 vector carrying the aurora A gene was transformed into E. coli DH10Bac cells containing the baculovirus genome (bacmid DNA) and via a transposition event in the cells, a region of the pFastBac vector containing gentamycin resistance gene and the aurora A gene including the baculovirus polyhedrin promoter was transposed directly into the bacmid DNA. By selection on gentamycin, kanamycin, tetracycline and X-gal, resultant white colonies should contain recombinant bacmid DNA encoding aurora A. Bacmid DNA was extracted from a small scale culture of several BH10Bac white colonies and transfected into Spodoptera frugiperda Sf21 cells grown in TC100 medium (GibcoBRL) containing 10% serum using CellFECTIN reagent (GibcoBRL) following manufacturer's instructions. Virus particles were harvested by collecting cell culture medium 72 hours post transfection. 0.5 ml of medium was used to infect 100 ml suspension culture of Sf21s containing 1×10⁷ cells/ml. Cell culture medium was harvested 48 hours post infection and virus titre determined using a standard plaque assay procedure. Virus stocks were used to infect Sf9 and “High 5” cells at a multiplicity of infection (MOI) of 3 to ascertain expression of recombinant aurora A protein.

For the large scale expression of aurora A kinase activity, Sf21 insect cells were grown at 28° C. in TC100 medium supplemented with 10% foetal calf serum (Viralex) and 0.2% F68 Pluronic (Sigma) on a Wheaton roller rig at 3 r.p.m. When the cell density reached 1.2×10⁶ cells ml⁻¹ they were infected with plaque-pure aurora A recombinant virus at a multiplicity of infection of 1 and harvested 48 hours later. All subsequent purification steps were performed at 4° C. Frozen insect cell pellets containing a total of 2.0×10⁸ cells were thawed and diluted with lysis buffer (25 mM HEPES (N-[2-hydroxyethyl]piperazine-N′-[2-ethanesulphonic acid]) pH 7.4 at 4° C., 100 mM KCl, 25 mM NaF, 1 mM Na₃VO₄, 1 mM PMSF (phenylmethylsulphonyl fluoride), 2 mM 2-mercaptoethanol, 2 mM imidazole, 1 μg/ml aprotinin, 1 μg/ml pepstatin, 1 μg/ml leupeptin), using 1.0 ml per 3×10⁷ cells. Lysis was achieved using a dounce homogeniser, following which the lysate was centrifuged at 41,000 g for 35 minutes. Aspirated supernatant was pumped onto a 5 mm diameter chromatography column containing 500 μl Ni NTA (nitrilo-tri-acetic acid) agarose (Qiagen, product no. 30250) which had been equilibrated in lysis buffer. A baseline level of UV absorbance for the eluent was reached after washing the column with 12 ml of lysis buffer followed by 7 ml of wash buffer (25 mM HEPES pH7.4 at 4° C., 100 mM KCl, 20 mM imidazole, 2 mM 2-mercaptoethanol). Bound aurora A protein was eluted from the column using elution buffer (25 mM HEPES pH7.4 at 4° C., 100 mM KCl, 400 mM imidazole, 2 mM 2-mercaptoethanol). An elution fraction (2.5 ml) corresponding to the peak in UV absorbance was collected. The elution fraction, containing active aurora A kinase, was dialysed exhaustively against dialysis buffer (25 mM HEPES pH7.4 at 4° C., 45% glycerol (v/v), 100 mM KCl, 0.25% Nonidet P40 (v/v), 1 mM dithiothreitol).

Each new batch of aurora A enzyme was titrated in the assay by dilution with enzyme diluent (25 mM Tris-HCl pH7.5, 12.5 mM KCl, 0.6 mM DTT). For a typical batch, stock enzyme is diluted 1 in 666 with enzyme diluent and 20 μl of dilute enzyme is used for each assay well. Test compounds (at 10 mM in dimethylsulphoxide (DMSO) were diluted with water and 10 μl of diluted compound was transferred to wells in the assay plates. “Total” and “blank” control wells contained 2.5% DMSO instead of compound. Twenty microlitres of freshly diluted enzyme was added to all wells, apart from “blank” wells. Twenty microlitres of enzyme diluent was added to “blank” wells. Twenty microlitres of reaction mix (25 mM Tris-HCl, 78.4 mM KCl, 2.5 mM NaF, 0.6 mM dithiothreitol, 6.25 mM MnCl₂, 25 mM ATP, 7.5 μM peptide substrate [biotin-LRRSLGLRRSLGLRRSLGLRRSLG]) containing 0.2 μCi [γ³³P]ATP (Amersham Pharmacia, specific activity ≧2500 Ci/mmol) was then added to all test wells to start the reaction. The plates were incubated at room temperature for 60 minutes. To stop the reaction 100 μl 20% v/v orthophosphoric acid was added to all wells. The peptide substrate was captured on positively-charged nitrocellulose P30 filtermat (Whatman) using a 96-well plate harvester (TomTek) and then assayed for incorporation of ³³P with a Beta plate counter. “Blank” (no enzyme) and “total” (no compound) control values were used to determine the dilution range of test compound which gave 50% inhibition of enzyme activity (IC₅₀ values). The compounds of the invention generally give IC₅₀ values of 0.05 nM to 10 μM.

### (b) In Vitro Aurora B Kinase Inhibition Test

This assay determines the ability of a test compound to inhibit serine-threonine kinase activity. DNA encoding aurora B may be obtained by total gene synthesis or by cloning. This DNA may then be expressed in a suitable expression system to obtain polypeptide with serine-threonine kinase activity. In the case of aurora B, the coding sequence was isolated from cDNA by polymerase chain reaction (PCR) and cloned into the pFastBac system in a manner similar to that described above for aurora A (i.e. to direct expression of a 6-histidine tagged aurora B protein).

For the large scale expression of aurora B kinase activity, Sf21 insect cells were grown at 28° C. in TC100 medium supplemented with 10% foetal calf serum (Viralex) and 0.2% F68 Pluronic (Sigma) on a Wheaton roller rig at 3 r.p.m. When the cell density reached 1.2×10⁶ cells ml⁻¹ they were infected with plaque-pure aurora B recombinant virus at a multiplicity of infection of 1 and harvested 48 hours later. All subsequent purification steps were performed at 4° C. Frozen insect cell pellets containing a total of 2.0×10⁸ cells were thawed and diluted with lysis buffer (50 mM HEPES (N-[2-hydroxyethyl]piperazine-N′-[2-ethanesulphonic acid]) pH7.5 at 4° C., 1 mM Na₃VO₄, 1 mM PMSF (phenylmethylsulphonyl fluoride), 1 mM dithiothreitol, 1 μg/ml aprotinin, 1 μg/ml pepstatin, 1 μg/ml leupeptin), using 1.0 ml per 2×10⁷ cells. Lysis was achieved using a sonication homogeniser, following which the lysate was centrifuged at 41,000 g for 35 minutes. Aspirated supernatant was pumped onto a 5 mm diameter chromatography column containing 1.0 ml CM sepharose Fast Flow (Amersham Pharmacia Biotech) which had been equilibrated in lysis buffer. A baseline level of UV absorbance for the eluent was reached after washing the column with 12 ml of lysis buffer followed by 7 ml of wash buffer (50 mM HEPES pH7.4 at 4° C., 1 mM dithiothreitol). Bound aurora B B protein was eluted from the column using a gradient of elution buffer (50 mM HEPES pH7.4 at 4° C., 0.6 M NaCl, 1 mM dithiothreitol, running from 0% elution buffer to 100% elution buffer over 15 minutes at a flowrate of 0.5 ml/min). Elution fractions (1.0 ml) corresponding to the peak in UV absorbance was collected. Elution fractions were dialysed exhaustively against dialysis buffer (25 mM HEPES pH7.4 at 4° C., 45% glycerol (v/v), 100 mM KCl, 0.05% (v/v) IGEPAL CA630 (Sigma Aldrich), 1 mM dithiothreitol). Dialysed fractions were assayed for aurora B kinase activity.

Each new batch of aurora B enzyme was titrated in the assay by dilution with enzyme diluent (25 mM Tris-HCl pH7.5, 12.5 mM KCl, 0.6 mM DTT). For a typical batch, stock enzyme is diluted 1 in 40 with enzyme diluent and 20 μl of dilute enzyme is used for each assay well. Test compounds (at 10 mM in dimethylsulphoxide (DMSO) were diluted with water and 10 μl of diluted compound was transferred to wells in the assay plates. “Total” and “blank” control wells contained 2.5% DMSO instead of compound. Twenty microlitres of freshly diluted enzyme was added to all wells, apart from “blank” wells. Twenty microlitres of enzyme diluent was added to “blank” wells. Twenty microlitres of reaction mix (25 mM Tris-HCl, 78.4 mM KCl, 2.5 mM NaF, 0.6 mM dithiothreitol, 6.25 mM MnCl₂, 37.5 mM ATP, 25 μM peptide substrate [biotin-LRRSLGLRRSLGLRRSLGLRRSLG]) containing 0.2 μCi [γ³³P]ATP (Amersham Pharmacia, specific activity ≧2500 Ci/mmol) was then added to all test wells to start the reaction. The plates were incubated at room temperature for 60 minutes. To stop the reaction 100 μl 20% v/v orthophosphoric acid was added to all wells. The peptide substrate was captured on positively-charged nitrocellulose P30 filtermat (Whatman) using a 96-well plate harvester (TomTek) & then assayed for incorporation of ³³P with a Beta plate counter. “Blank” (no enzyme) and “total” (no compound) control values were used to determine the dilution range of test compound which gave 50% inhibition of enzyme activity (IC₅₀ values). The compounds of the invention generally give IC₅₀ values of 0.05 nM to 10 μM

### (c) In Vitro Cell Phenotype and Substrate Phosphorylation Assay

This assay is used to determine the cellular effects of compounds on SW620 human colon tumour cells in vitro. Compounds typically cause inhibition of levels of phosphohistone H3 and an increase in the nuclear area of the cells.

10⁴ SW620 cells per well were plated in 100 μl DMEM media (containing 10% FCS and 1% glutamine) (DMEM is Dulbecco's Modified Eagle's Medium (Sigma D6546)) in costar 96 well plates and left overnight at 37° C. and 5% CO₂ to adhere. The cells were then dosed with compound diluted in media (50 μl is added to each well to give 0.00015μ-1 μM concentrations of compound) and after 24 hours of treatment with compound, the cells were fixed.

The cells were first examined using a light microscope and any cellular changes in morphology were noted. 100 μl of 3.7% formaldehyde was then added to each well, and the plate was left for at least 30 minutes at room temperature. Decanting and tapping the plate on a paper towel removed the fixative and plates were then washed once in PBS (Dulbecco's Phosphate Buffered Saline (Sigma D8537)) using an automated plate washer. 100 μl PBS and 0.5% triton X-100 was added and the plates were put on a shaker for 5 minutes. The plates were washed in 100 μl PBS and solution tipped off. 50 μl of primary antibody, 1:500 rabbit anti-phosphohistone H3 in PBS 1% BSA (bovine serum albumin) and 0.5% tween, was added. Anti-phosphohistone H3 rabbit polyclonal 06-750 was purchased from Upstate Biotechnology. The plates were left 1 hour at room temperature on a shaker.

The next day, the antibody was tipped off and the plates were washed twice with PBS. In an unlit area, 50 μl of secondary antibody, 1:10,000 Hoechst and 1:200 Alexa Fluor 488 goat anti rabbit IgGA (cat no. 11008 molecular probes) in PBS 1% BSA, 0.5% tween was added. The plates were wrapped in tin foil and shaken for 1 hour at room temperature. The antibody was tipped off and plates were washed twice with PBS. 200 μl PBS was added to each well, and the plates were shaken for 10 minutes, PBS was removed. 100 μl PBS was added to each well and the plates were sealed ready to analyse. Analysis was carried out using an Arrayscan Target Activation algorithm to measure cellular levels of phosphohistone H3 and changes in nuclear area. Results were reported as the effective concentration required to give 50% inhibition of phosphohistone H3 levels and similarly for a 50% increase in nuclear area of cells (EC50 values). The compounds of the invention generally give EC50 values for inhibition of phosphohistone H3 levels of 0.5 nM to 1 μM and in particular compound 9 of table 2 had an EC50 value of 7.6 nM.

### (d) In Vitro Drug-Resistant Cell Phenotype and Substrate Phosphorylation Assay.

This assay is used to determine the cellular effects of compounds on drug-resistant MCF7-ADR human breast tumour cells in vitro.

MCF7 cells were pretreated with multiple doses of adriomycin (Dr. Hickinson, Molecular Oncology lab, ICRF, University of Oxford Institute of Molecular Medicine, Headington, Oxford), a procedure that resulted in overexpression of drug-resistant proteins by the cells. Compounds typically cause inhibition of levels of phosphohistoneH3 and an increase in the nuclear area of treated cells. However, if the compounds are substrates of the overexpressed efflux proteins, they will appear less active in this assay than in the previous SW620 assay.

0.8×10⁴ MCF7-ADR cells per well were plated in 100 μl DMEM media (containing 10% FCS (foetal calf serum) and 1% glutamine) in costar 96 well plates and left overnight at 37° C. and 5% CO₂ to adhere.

All other procedures are identical to those for the above assay using SW620 cells.

The compounds of the invention generally have EC50 values for inhibition of phosphohistone H3 levels of 0.5 nM to 1 μM, and in particular compound 9 of table 2 had an EC50 value of 0.4 μM.

### (e) Plasma Protein Binding Assay

This assay is used to determine the extent to which compounds bind to plasma proteins and hence free drug levels in plasma.

Plasma protein binding was measured using the equilibrium dialysis technique. Compound was added to human plasma to a concentration of 20 μM and dialysed with isotonic phosphate/sodium chloride buffer for 18 hours at 37° C. The plasma and buffer solutions on each side of the dialysis membrane were analysed using a generic LCUVMS (Waters 2795 HPLC with Micromass ZQ mass spectrometer) and the percentage of free compound in 100% plasma was determined.

The compounds of the invention typically have a percentage of free compound in 100% plasma of 0.5 to 50% and in particular compound 9 of table 2 had a value of 3.9%.

The invention will now be illustrated in the following examples, in which standard techniques known to the skilled chemist and techniques analogous to those described in these examples may be used where appropriate, and in which, unless otherwise stated:

(i) evaporations were carried out by rotary evaporation in vacuo and work up procedures were carried out after removal of residual solids such as drying agents by filtration;
(ii) operations were carried out at ambient temperature, typically in the range 18-25° C. and in air unless stated, or unless the skilled person would otherwise operate under an atmosphere of an inert gas such as argon;
(iii) column chromatography (by the flash procedure) and medium pressure liquid chromatography (MPLC) were performed on Merck Kieselgel silica (Art. 9385);
(iv) yields are given for illustration only and are not necessarily the maximum attainable;
(v) the structures of the end products of the formula (I) were generally confirmed by nuclear (generally proton) magnetic resonance (NMR) and mass spectral techniques; proton magnetic resonance chemical shift values were measured in deuterated dimethyl sulphoxide (DMSO d₆) (unless otherwise stated) on the delta scale (ppm downfield from tetramethylsilane) using one of the following four instruments
Varian Gemini 2000 spectrometer operating at a field strength of 300 MHz
Bruker DPX300 spectrometer operating at a field strength of 300 MHz
JEOL EX 400 spectrometer operating at a field strength of 400 MHz
Bruker Avance 500 spectrometer operating at a field strength of 500 MHz
   Peak multiplicities are shown as follows: s, singlet; d, doublet; dd, double doublet; t, triplet; q, quartet; qu, quintet; m, multiplet; br s, broad singlet;
   (vi) robotic synthesis was carried out using a Zymate XP robot, with solution additions via a Zymate Master Laboratory Station and stirred via a Stem RS5000 Reacto-Station at 25° C.;
   (vii) work up and purification of reaction mixtures from robotic synthesis was carried out as follows: evaporations were carried out in vacuo using a Genevac HT 4; column chromatography was performed using either an Anachem Sympur MPLC system on silica using 27 mm diameter columns filled with Merck silica (60 μm, 25 g); the structures of the final products were confirmed by LCMS (liquid chromatography mass spectrometry) on a Waters 2890/ZMD micromass system using the following and are quoted as retention time (RT) in minutes:

| | |
|---|---|
| Column: | waters symmetry C18 3.5 μm 4.6 × 50 mm |
| Solvent A: | H₂O |
| Solvent B: | CH₃CN |
| Solvent C: | MeOH + 5% HCOOH |
| Flow rate: | 2.5 ml/min |
| Run time: | 5 minutes with a 4.5 minute gradient from 0-100% C |
| Wavelength: | 254 nm, bandwidth 10 nm |
| Mass detector: | ZMD micromass |
| Injection volume | 0.005 ml |

(viii) Analytical LCMS for compounds which had not been prepared by robotic synthesis was performed on a Waters Alliance HT system using the following and are quoted as retention time (RT) in minutes:

| | |
|---|---|
| Column: | 2.0 mm × 5 cm Phenomenex Max-RP 80A |
| Solvent A: | Water |
| Solvent B: | Acetonitrile |
| Solvent C: | Methanol/1% formic acid or Water/1% formic acid |
| Flow rate: | 1.1 ml/min |
| Run time: | 5 minutes with a 4.5 minute gradient from 0-95% B + |
| | constant 5% solvent C |
| Wavelength: | 254 nm, bandwidth 10 nm |
| Injection volume | 0.005 ml |
| Mass detector: | Micromass ZMD |

(ix) Preparative high performance liquid chromatography (HPLC) was performed on either
Waters preparative LCMS instrument, with retention time (RT) measured in minutes:

| | |
|---|---|
| Column: | β-basic Hypercil (21 × 100 mm) 5 μm |
| Solvent A: | Water/0.1% Ammonium carbonate |
| Solvent B: | Acetonitrile |
| Flow rate: | 25 ml/min |
| Run time: | 10 minutes with a 7.5 minute gradient from 0-100% B |
| Wavelength: | 254 nm, bandwidth 10 nm |
| Injection volume | 1-1.5 ml |
| Mass detector: | Micromass ZMD |

Gilson preparative HPLC instrument, with retention time (RT) measured in minutes:

| | |
|---|---|
| Column: | 21 mm × 15 cm Phenomenex Luna2 C18 |
| Solvent A: | Water + 0.1% trifluoracetic acid, |
| Solvent B: | Acetonitrile + 0.1% trifluoracetic acid |
| Flow rate: | 21 ml/min |
| Run time: | 20 minutes with various 10 minute gradients from |
| | 5-100% B |
| Wavelength: | 254 nm, bandwidth 10 nm |
| Injection volume | 0.1-4.0 ml |

(x) intermediates were not generally fully characterised and purity was assessed by thin layer chromatography (TLC), HPLC, infra-red (IR), MS or NMR analysis.

TABLE 1

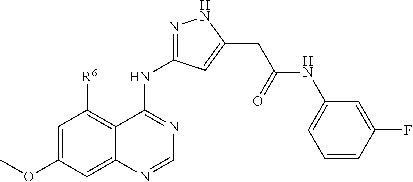

| Example | R⁶ |
|---|---|
| 1 | 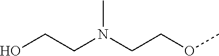 |
| 2 | 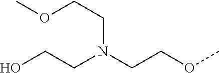 |
| 3 | 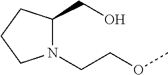 |
| 4 | 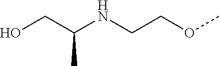 |

TABLE 2

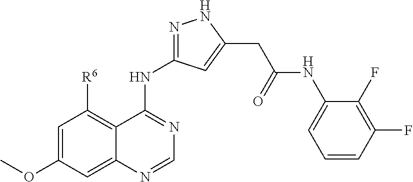

| Example | R⁶ |
|---|---|
| 5 | 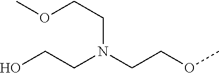 |
| 6 | 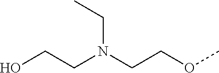 |
| 7 | 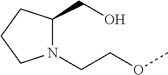 |
| 8 | 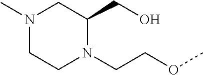 |
| 9 | 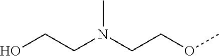 |

TABLE 3

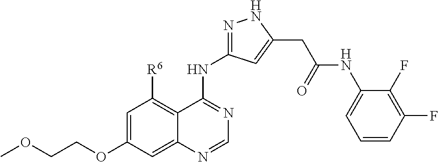

| Example | R⁶ |
|---|---|
| 10 | 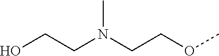 |
| 11 | 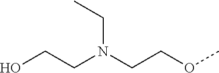 |
| 12 | 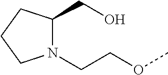 |

TABLE 4

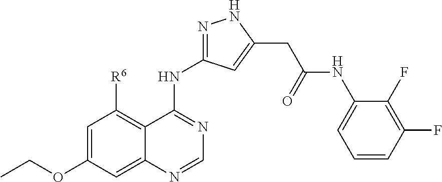

| Example | R⁶ |
|---|---|
| 13 | 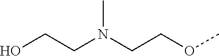 |
| 14 | 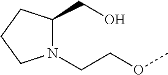 |
| 15 | 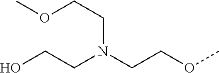 |

TABLE 5

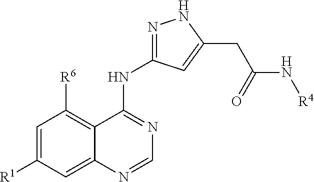

| Example | R⁶ | R¹ | R⁴ |
|---|---|---|---|
| 16 | 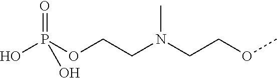 | 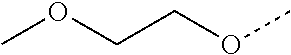 | 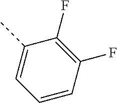 |
| 17 | 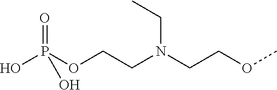 | 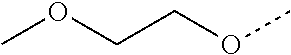 | 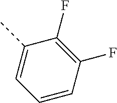 |
| 18 | 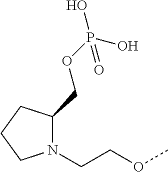 | 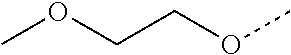 | 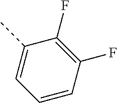 |
| 19 | 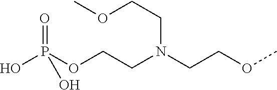 | MeO | 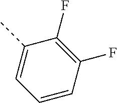 |
| 20 | 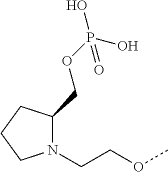 | MeO | 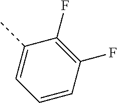 |
| 21 | 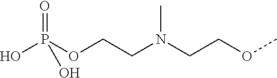 | MeO | 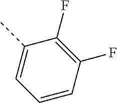 |
| 22 | 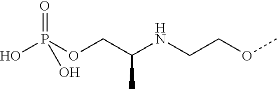 | MeO | 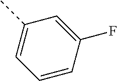 |

TABLE 6

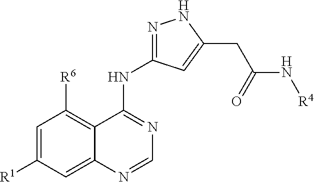

| Example | R¹ | R⁴ | R⁶ |
|---|---|---|---|
| 23 | 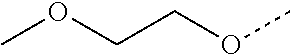 | 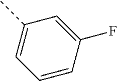 | 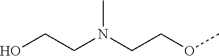 |
| 24 | EtO | 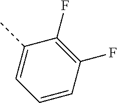 | 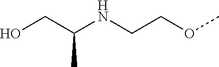 |
| 25 | EtO | 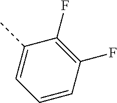 | 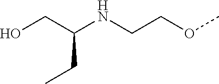 |
| 26 | EtO | 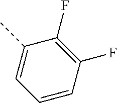 | 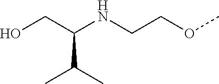 |
| 27 | EtO | 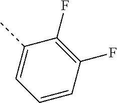 | 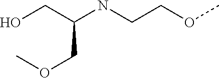 |
| 28 | MeO | 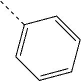 | 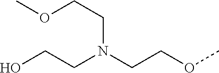 |
| 29 | MeO | 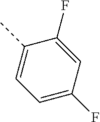 | 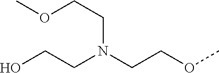 |
| 30 | MeO | 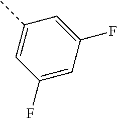 | 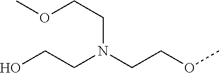 |
| 31 | MeO | 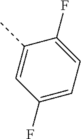 | 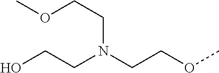 |
| 32 | 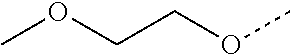 | 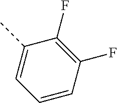 | 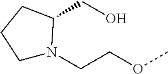 |
| 33 | 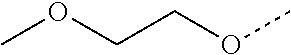 | 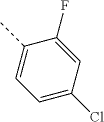 | 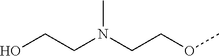 |
| 34 | 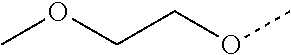 | 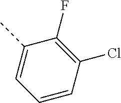 | 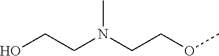 |

### EXAMPLE 1

### Preparation of Compound 1 in Table 1

### N-(3-fluorophenyl)-2-{3-[(5-[2-[(2-hydroxyethyl)(methyl)amino]ethoxy}-7-methoxyquinazolin-4-yl)amino]-1H-pyrazol-5-yl]acetamide

A solution of 2-(3-{[5-(2-chloroethoxy)-7-methoxyquinazolin-4-yl]amino}-1H-pyrazol-5-yl)-N-(3-fluorophenyl)acetamide (0.201 g, 0.43 mmol), 2-(methylamino)ethanol (0.50 ml, 6.2 mmol) and potassium iodide (0.141 g, 0.85 mmol) in dimethylacetamide (4 ml) was heated at 85° C. for 3 hours. The mixture was made acidic by the addition of trifluoroacetic acid and then diluted with a mixture of di-methylsulphoxide:acetonitrile:water 7:3:1 (6 ml) and then purified directly by reverse phase chromatography eluting with a gradient of 5 to 50% acetonitrile, containing 0.2% trifluoroacetic acid, in water, containing 0.2% trifluoroacetic acid. Fractions containing the product were combined and made basic by the addition of sodium hydrogencarbonate and then the mixture was concentrated in vacuo. The mixture was filtered and the residue was washed with water and then diethylether to leave compound 1 in table 1 (0.092 g, 42% yield):

¹H-NMR (DMSO d₆, TFA): 10.66 (s, 1H), 10.51 (s, 1H), 9.90 (br s, 1H), 8.91 (s, 1H), 7.65-7.59 (m, 1H), 7.41-7.29 (m, 2H), 7.05 (d, 1H), 6.95 (d, 1H), 6.93-6.87 (m, 1H), 6.77 (s, 1H), 4.81 (br s, 2H), 4.00 (s, 3H), 3.99-3.70 (m, 6H), 3.50-3.28 (m, 2H), 3.00 (s, 3H).

MS (+ve ESI): 510 (M+H)⁺

2-(3-{[5-(2-chloroethoxy)-7-methoxyquinazolin-4-yl]amino}-1H-pyrazol-5-yl)-N-(3-fluorophenyl)acetamide used as starting material was prepared as follows:

a) A solution of 5,7-dimethoxyquinazolin-4(3H)-one (5.0 g, 24 mmol) (for synthesis see: Hennequin, Laurent Francois Andre; Ple, Patrick. Preparation of 4-anilinoquinazoline derivatives for the treatment of tumors. PCT Int. Appl. (2001), WO 01/094341), di-iso-propylethylamine (13.5 ml, 77.7 mmol) and phosphorus oxychloride (7.24 ml, 77.7 mmol) in acetonitrile (125 ml) was heated under reflux for 2 hours. The mixture was evaporated in vacuo and the residue was partitioned between dichloromethane and a saturated solution of sodium hydrogencarbonate. The organic layer was separated and washed with a solution of brine, dried over magnesium sulphate and then evaporated to leave a dark brown solid. The crude product was purified by flash chromatography on silica eluting with ethylacetate to give 4-chloro-5,7-dimethoxyquinazoline (2.83 g, 52% yield):

¹H-NMR (DMSO d₆): 8.81 (s, 1H), 7.01 (d, 1H), 6.83 (d, 1H), 3.96 (s, 3H), 3.95 (s, 3H),

MS (+ve ESI): 225/227 (M+H)⁺

b) A mixture of 4-chloro-5,7-dimethoxyquinazoline (2.9 g, 11.9 mmol) and 2-(3-amino-1H-pyrazol-5-yl)-N-(3-fluorophenyl)acetamide (2.72 g, 11.6 mmol) in iso-propylalcohol (75 ml) was heated at 90° C. for 2 hours. The mixture was allowed to cool to room temperature and then diethyl ether was added to give a fine solid. The mixture was filtered and washed with diethylether and then dried in air to leave 2-{3-[(5,7-dimethoxyquinazolin-4-yl)amino]-1H-pyrazol-5-yl}-N-(3-fluorophenyl)acetamide as the hydrochloride salt (4.95 g, 91% yield):

¹H-NMR (DMSO d₆): 10.72 (s, 1H), 10.71 (s, 1H), 8.83 (s, 1H), 7.66-7.61 (m, 1H), 7.40-7.32 (m, 2H), 6.97-6.94 (m, 2H), 6.92-6.86 (m, 1H), 6.71 (s, 1H), 4.16 (s, 3H), 3.97 (s, 3H), 3.84 (s, 2H).

MS (+ve ESI): 423 (M+H)⁺

c) A mixture of 2-{3-[(5,7-dimethoxyquinazolin-4-yl)amino]-1H-pyrazol-5-yl}-N-(3-fluorophenyl)acetamide hydrochloride (4.95 g, 10.8 mmol) and pyridine hydrochloride (6.93 g, 60 mmol) in pyridine (60 ml) was heated at 110° C. for 6.5 hours. The mixture was allowed to cool to room temperature and then poured into water (350 ml) and neutralised with a solution of ammonia. The resulting suspension was filtered and the residue was washed with water, acetonitrile and finally with diethyl ether and the solid dried in vacuo to leave N-(3-fluorophenyl)-2-{3-[(5-hydroxy-7-methoxyquinazolin-4-yl)amino]-1H-pyrazol-5-yl}acetamide (3.6 g, 82% yield):

¹H-NMR (DMSO d₆): 10.38 (s, 1H), 8.20 (s, 1H), 7.65-7.58 (m, 1H), 7.39-7.30 (m, 2H), 6.91-6.84 (m, 1H), 6.56 (br s, 1H), 6.10 (s, 1H), 6.05 (s, 1H), 3.73 (s, 3H), 3.69 (s, 2H).

MS (+ve ESI): 409 (M+H)⁺

d) Di-tert-butylazodicarboxylate (1.29 g, 5.59 mmol) was added to a solution of N-(3-fluorophenyl)-2-{3-[(5-hydroxy-7-methoxyquinazolin-4-yl)amino]-1H-pyrazol-5-yl}acetamide (1.9 g, 4.7 mmol), triphenylphosphine (1.46 g, 5.59 mmol) and 2-chloroethanol (0.437 ml, 6.52 mmol) in tetrahydrofuran (45 ml). The mixture was stirred at room temperature for 20 minutes and then further portions of triphenylphosphine (1.46 g, 5.59 mmol) and di-tert-butylazodicarboxylate (1.29 g, 5.59 mmol) were added. The mixture was stirred at room temperature for 3 hours and then further portions of triphenylphosphine (1.46 g, 5.59 mmol) and di-tert-butylazodicarboxylate (1.29 g, 5.59 mmol) were added and the mixture stirred at room temperature for 1.5 hours. Diethyl ether was added to cause precipitation of the product. The mixture was filtered and the residue was washed with diethyl ether, followed by water, and then acetonitrile and finally again with diethyl ether to leave 2-(3-{[5-(2-chloroethoxy)-7-methoxyquinazolin-4-yl]amino}-1H-pyrazol-5-yl)-N-(3-fluorophenyl)acetamide (1.21 g, 56% yield):

MS (+ve ESI): 471/473 (M+H)⁺

2-(3-amino-1H-pyrazol-5-yl)-N-(3-fluorophenyl)acetamide was prepared as follows:

i) Pentafluorophenyl trifluoroacetate (48.9 ml, 284 mmol) was added dropwise, over 40 minutes maintaining the internal temperature below 15° C., to a solution of (3-amino-1H-pyrazol-5-yl)acetic acid (20.0 g, 142 mmol) and pyridine (25.3 ml, 313 mmol) in dimethylformamide (200 ml). The mixture was then allowed to warm to room temperature and stirred for 1.5 hours. 3-Fluoroaniline (31.6 g, 284 mmol) was added in a single portion and the mixture was then stirred at room temperature for 1.5 hours. The mixture was poured into a dilute solution of hydrochloric acid and the resultant solid was filtered, washed with water and then diethyl ether to leave 2,2,2-trifluoro-N-(5-{2-[(3-fluorophenyl)amino]-2-oxoethyl}-1H-pyrazol-3-yl)acetamide (33.3 g, 71% yield).

MS (+ve ESI): 331 (M+H)⁺

ii) A mixture of 2,2,2-trifluoro-N-(5-{2-[(3-fluorophenyl)amino]-2-oxoethyl}-1H-pyrazol-3-yl)acetamide (33.3 g, 100 mmol) in methanol (250 ml) and hydrochloric acid (2M, 250 ml) was heated at 40° C. for 2.5 hours. The mixture was neutralised with sodium hydrogencarbonate and then the mixture was concentrated in vacuo to approximately ⅓ volume. The resultant precipitate was filtered and the residue washed carefully with ice-cold water and then allowed to dry in air to leave 2-(3-amino-1H-pyrazol-5-yl)-N-(3-fluorophenyl)acetamide (12.2 g, 52% yield):

MS (+ve ESI): 235 (M+H)⁺

### EXAMPLE 2

### Preparation of Compound 2 in Table 1

### N-(3-fluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide

Prepared in an analogous way to that described for example 1 in table 1 using 2-[(2-methoxyethyl)amino]ethanol (0.252 g, 2.1 mmol) and 2-(3-{[5-(2-chloroethoxy)-7-methoxyquinazolin-4-yl]amino}-1H-pyrazol-5-yl)-N-(3-fluorophenyl)acetamide (0.15 g, 0.32 mmol), to give compound 2 in table 1 (0.080 g, 45% yield):

¹H-NMR (DMSO d₆): 12.4 (s, 1H), 10.18 (s, 1H), 8.42 (s, 1H), 7.60 (d, 1H), 7.35 (m, 2H), 6.89 (t, 1H), 6.78 (d, 1H), 6.71 (m, 2H), 4.32 (t, 2H), 3.89 (s, 3H), 3.71 (s, 2H), 3.50 (t, 2H), 3.32 (t, 2H), 3.10 (s, 3H), 3.05 (t, 2H), 2.75 (t, 2H), 2.69 (t, 2H).

MS (+ve ESI): 554 (M+H)⁺

### EXAMPLE 3

### Preparation of Compound 3 in Table 1

### N-(3-fluorophenyl)-2-{3-[(5-{2-[(2S)-2-(hydroxymethyl)pyrrolidin-1-yl]ethoxy}-7-methoxyquinazolin-4-yl)amino]-1H-pyrazol-5-yl}acetamide

Prepared in an analogous way to that described for example 1 in table 1 using (2S)-pyrrolidin-2-ylmethanol (0.214 g, 2.1 mmol) and 2-(3-{[5-(2-chloroethoxy)-7-methoxyquinazolin-4-yl]amino}-1H-pyrazol-5-yl)-N-(3-fluorophenyl)acetamide (0.15 g, 0.32 mmol), to give compound 3 in table 1 (0.073 g, 43% yield):

¹H-NMR (DMSO d₆): 10.15 (s, 1H), 8.43 (s, 1H), 7.60 (d, 1H), 7.35 (m, 2H), 6.88 (t, 1H), 6.78 (s, 1H), 6.72 (m, 2H), 4.35 (m, 3H), 3.88 (s, 3H), 3.72 (s, 2H), 3.40 (m, 4H), 2.75 (m, 1H), 2.52 (m, 1H), 2.35 (m, 1H), 1.75 (m, 1H), 1.55 (m, 2H).

MS (+ve ESI): 536 (M+H)⁺

### EXAMPLE 4

### Preparation of Compound 4 in Table 1

### N-(3-fluorophenyl)-2-(3-{[5-(2-{[(1S)-2-hydroxy-1-methylethyl]amino}ethoxy)-7-methoxyquinazolin-4-yl]amino}-1H-pyrazol-5-yl)acetamide

Prepared in an analogous way to that described for example 1 in table 1 using (2S)-2-aminopropan-1-ol (0.159 g, 2.1 mmol) and 2-(3-{[5-(2-chloroethoxy)-7-methoxyquinazolin-4-yl]amino}-1H-pyrazol-5-yl)-N-(3-fluorophenyl)acetamide (0.15 g, 0.32 mmol), to give compound 4 in table 1 (0.050 g, 31% yield):

¹H-NMR (DMSO d₆): 12.35 (s, 1H), 10.39 (s, 1H), 10.30 (s, 1H), 8.40 (s, 1H), 7.60 (d, 1H), 7.35 (m, 2H), 6.88 (t, 1H), 6.80 (s, 1H), 6.75 (s, 1H), 6.65 (s, 1H), 4.62 (app.s, 1H), 4.25 (app.s, 2H), 3.89 (s, 3H), 3.75 (s, 2H), 3.65 (m, 2H), 3.04 (app.s, 2H), 1.00 (d, 3H).

MS (+ve ESI): 510 (M+H)⁺

### EXAMPLE 5

### Preparation of Compound 5 in Table 2

### N-(2,3-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide

Prepared in an analogous way to that described in example 1 in table 1 using 2-[(2-methoxyethyl)amino]ethanol (0.305 g, 2.56 mmol) and 2-(3-{[5-(2-chloroethoxy)-7-methoxyquinazolin-4-yl]amino}-1H-pyrazol-5-yl)-N-(2,3-difluorophenyl)acetamide (0.25 g, 0.51 mmol), to give compound 5 in table 2 (0.080 g, 27% yield):

¹H-NMR (DMSO d₆): 10.19 (s, 1H), 8.45 (s, 1H), 7.75-7.68 (m, 1H), 7.24-7.14 (m, 2H), 6.79 (d, 1H), 6.80-6.73 (m, 1H), 6.74 (d, 1H), 4.34 (t, 2H), 3.90 (s, 3H), 3.83 (s, 2H), 3.49 (t, 2H), 3.38 (t, 2H partially obscured by water), 3.35-3.17 (water), 3.11 (s, 3H), 3.06 (t, 2H), 2.77 (t, 2H), 2.69 (t, 2H).

MS (+ve ESI): 572 (M+H)⁺

2-(3-{[5-(2-chloroethoxy)-7-methoxyquinazolin-4-yl]amino}-1H-pyrazol-5-yl)-N-(2,3-difluorophenyl)acetamide used as starting material was prepared as follows:

a) A mixture of 4-chloro-5,7-dimethoxyquinazoline (3.0 g, 13.4 mmol) and 2-(3-amino-1H-pyrazol-5-yl)-N-(2,3-difluorophenyl)acetamide (3.20 g, 12.7 mmol) in iso-propylalcohol (80 ml) was heated at 90° C. for 30 minutes. A further portion of 2-(3-amino-1H-pyrazol-5-yl)-N-(2,3-difluorophenyl)acetamide (ca. 0.3 g) was added and the mixture heated at 90° C. for 15 minutes. The mixture was allowed to cool to room temperature and then diethyl ether was added to give a fine solid. The mixture was filtered and washed with diethylether and then dried in vacuo to leave N-(2,3-difluorophenyl)-2-{3-[(5,7-dimethoxyquinazolin-4-yl)amino]-1H-pyrazol-5-yl}acetamide as the hydrochloride salt (5.79 g, 96% yield):

¹H-NMR (DMSO d₆): 10.67 (s, 1H), 10.33 (br s, 1H), 8.81 (s, 1H), 7.71-7.64 (m, 1H), 7.25-7.14 (m, 2H), 6.98 (br s, 1H), 6.94 (d, 1H), 6.73 (s, 1H), 4.16 (s, 3H), 3.97 (s, 3H), 3.91 (s, 2H).

MS (+ve ESI): 441 (M+H)⁺

b) A mixture of N-(2,3-difluorophenyl)-2-{3-[(5,7-dimethoxyquinazolin-4-yl)amino]-1H-pyrazol-5-yl}acetamide hydrochloride (9.06 g, 19.0 mmol) and pyridine hydrochloride (12.7 g, 110 mmol) in pyridine (110 ml) was heated at 110° C. for a total of 8 hours. The mixture was allowed to cool to room temperature and then poured into a saturated solution of sodium bicarbonate (1000 ml). The resulting suspension was filtered and the residue was washed with water, acetonitrile and finally with diethyl ether and the solid dried in vacuo to leave N-(2,3-difluorophenyl)-2-{3-[(5-hydroxy-7-methoxyquinazolin-4-yl)amino]-1H-pyrazol-5-yl}acetamide (6.78 g, 84% yield):

¹H-NMR (DMSO d₆): 10.16 (s, 1H), 8.22 (s, 1H), 7.75-7.68 (m, 1H), 7.23-7.14 (m, 2H), 6.58 (br s, 1H), 6.15 (br s, 1H), 6.11 (br s, 1H), 3.79 (s, 2H), 3.75 (s, 3H).

MS (+ve ESI): 427 (M+H)⁺

c) Di-tert-butylazodicarboxylate (2.79 g, 12.1 mmol) was added to a solution of N-(2,3-difluorophenyl)-2-{3-[(5-hydroxy-7-methoxyquinazolin-4-yl)amino]-1H-pyrazol-5-yl}acetamide (4.3 g, 10.1 mmol), triphenylphosphine (30.17 g, 12.1 mmol) and 2-chloroethanol (0.944 ml, 14.1 mmol) in tetrahydrofuran (100 ml). The mixture was stirred at room temperature for 30 minutes and then further portions of triphenylphosphine (3.17 g, 12.1 mmol) and di-tert-butylazodicarboxylate (2.79 g, 12.1 mmol) were added. The mixture was stirred at room temperature for 3 hours and then diethyl ether was added to cause precipitation of the product. The mixture was filtered and the residue was washed with diethyl ether, followed by water, and then acetonitrile and finally again with diethyl ether to leave 2-(3-{[5-(2-chloroethoxy)-7-methoxyquinazolin-4-yl]amino}-1H-pyrazol-5-yl)-N-(2,3-difluorophenyl)acetamide (3.54 g, 72% yield):

MS (+ve ESI): 489/491 (M+H)⁺

2-(3-amino-1H-pyrazol-5-yl)-N-(2,3-difluorophenyl)acetamide was prepared as follows:

i) Pentafluorophenyl trifluoroacetate (24.4 ml, 142 mmol) was added dropwise, over 30 minutes maintaining the internal temperature below 15° C., to a solution of (3-amino-1H-pyrazol-5-yl)acetic acid (10.0 g, 71 mmol) and pyridine (12.6 ml, 156 mmol) in dimethylformamide (100 ml). The mixture was then allowed to warm to room temperature and stirred for 30 minutes. 2,3-Difluoroaniline (18.3 g, 142 mmol) was added in a single portion and the mixture was then stirred at room temperature for 3 hours. The mixture was heated at 90° C. for 2 hours. The mixture was poured into a dilute solution of hydrochloric acid and the resultant solid was filtered, washed with water and then toluene and iso-hexane to leave N-(5-{2-[(2,3-difluorophenyl)amino]-2-oxoethyl}-1H-pyrazol-3-yl)-2,2,2-trifluoroacetamide (18.0 g, 74% yield).

MS (−ve ESI): 347 (M−H)⁻

ii) A mixture of N-(5-{2-[(2,3-difluorophenyl)amino]-2-oxoethyl}-1H-pyrazol-3-yl)-2,2,2-trifluoroacetamide (25.9 g, 74.5 mmol) in methanol (200 ml) and hydrochloric acid (2M, 200 ml) was heated at 50° C. for 3 hours. The mixture was neutralised with sodium hydrogencarbonate and then the mixture was concentrated in vacuo to approximately ⅓ volume. The resultant precipitate was filtered and the residue washed carefully with ice-cold water and then dried in vacuo to leave 2-(3-amino-1H-pyrazol-5-yl)-N-(2,3-difluorophenyl)acetamide (14.01 g, 75% yield):

MS (+ve ESI): 253 (M+H)⁺

### EXAMPLE 6

### Preparation of Compound 6 in Table 2

### N-(2,3-difluorophenyl)-2-{3-[(5-{2-[ethyl(2-hydroxyethyl)amino]ethoxy}-7-methoxyquinazolin-4-yl)amino]-1H-pyrazol-5-yl}acetamide

Prepared in an analogous way to that described in example 5 in table 2 using 2-(ethylamino)ethanol (0.182 g, 2.05 mmol) and 2-(3-{[5-(2-chloroethoxy)-7-methoxyquinazolin-4-yl]amino}-1H-pyrazol-5-yl)-N-(2,3-difluorophenyl)acetamide (0.20 g, 0.41 mmol), to give compound 6 in table 2 (0.093 g, 42% yield):

¹H-NMR (DMSO d₆): 10.23 (s, 1H), 10.18 (s, 1H), 8.45 (s, 1H), 7.75-7.67 (m, 1H), 7.24-7.14 (m, 2H), 6.79 (d, 1H), 6.75 (d, 1H), 4.34 (t, 2H), 3.90 (s, 3H), 3.83 (s, 2H), 3.49 (t, 2H), 2.97 (t, 2H), 2.65 (q, 2H), 2.62 (t, 2H), 0.93 (t, 3H).

MS (+ve ESI): 542 (M+H)⁺

### EXAMPLE 7

### Preparation of Compound 7 in Table 2

### N-(2,3-difluorophenyl)-2-{3-[(5-{2-[(2S)-2-(hydroxymethyl)pyrrolidin-1-yl]ethoxy}-7-methoxyquinazolin-4-yl)amino]-1H-pyrazol-5-yl}acetamide

Prepared in an analogous way to that described in example 5 in table 2 using (2S)-pyrrolidin-2-ylmethanol (0.156 g, 1.54 mmol) and 2-(3-{[5-(2-chloroethoxy)-7-methoxyquinazolin-4-yl]amino}-1H-pyrazol-5-yl)-N-(2,3-difluorophenyl)acetamide (0.25 g, 0.51 mmol), to give compound 7 in table 2 (0.075 g, 26% yield):

¹H-NMR (DMSO d₆): 10.19 (s, 1H), 10.17 (s, 1H), 8.45 (s, 1H), 7.76-7.66 (m, 1H), 7.24-7.14 (m, 2H), 6.79 (d, 1H), 6.81-6.72 (m, 1H), 6.74 (d, 1H), 4.43-4.28 (m, 2H), 3.90 (s, 3H), 3.83 (s, 2H), 3.46-3.25 (under water), 3.20-3.12 (m, 1H), 2.80-2.72 (m, 1H), 2.60-2.50 (under DMSO), 2.35-2.27 (m, 1H), 1.82-1.71 (m, 1H), 1.65-1.48 (m, 3H).

MS (+ve ESI): 554 (M+H)⁺

### EXAMPLE 8

### Preparation of Compound 8 in Table 2

### N-(2,3-difluorophenyl)-2-{3-[(5-{2-[(2)-2-(hydroxymethyl)-4-methylpiperazin-1-yl]ethoxy}-7-methoxyquinazolin-4-yl)amino]-1H-pyrazol-5-yl}acetamide

Prepared in an analogous way to that described in example 5 in table 2 using [(2S)-4-methylpiperazin-2-yl]methanol (0.333 g, 2.56 mmol) (see: Falorni, M; Satta, S; Conti, S; Glacomelli, G, Tetrahedron Asymmetry, 1993, 4(11), 2389-2398) and 2-(3-{[5-(2-chloroethoxy)-7-methoxyquinazolin-4-yl]amino}-1H-pyrazol-5-yl)-N-(2,3-difluorophenyl)acetamide (0.25 g, 0.51 mmol), to give compound 8 in table 2 (0.082 g, 28% yield):

¹H-NMR (DMSO d₆): 12.38 (s, 1H), 10.17 (s, 1H), 10.12 (s, 1H), 8.43 (s, 1H), 7.77-7.68 (m, 1H), 7.24-7.14 (m, 2H), 6.83-6.72 (m, 3H), 4.40-4.32 (m, 2H), 3.90 (s, 3H), 3.84 (s, 2H), 3.66-3.58 (m, 1H), 3.53-3.37 (m, 2H), 3.28 (s, 3H), 2.93-2.84 (m, 1H), 2.80-2.66 (m, 1H), 2.53-2.30 (m, 3H partially under DMSO), 2.07-1.93 (m, 3H).

MS (+ve ESI): 583 (M+H)⁺

### EXAMPLE 9

### Preparation of Compound 9 in Table 2

### N-(2,3-difluorophenyl)-2-{3-[(5-{2-[(2-hydroxyethyl)(methyl)amino]ethoxy}-7-methoxyquinazolin-4-yl)amino]-1H-pyrazol-5-yl}acetamide

Prepared in an analogous way to that described in example 5 in table 2 using 2-(methylamino)ethanol (0.098 g, 1.3 mmol) and 2-(3-{[5-(2-chloroethoxy)-7-methoxyquinazolin-4-yl]amino}-1H-pyrazol-5-yl)-N-(2,3-difluorophenyl)acetamide (0.16 g, 0.33 mmol), to give compound 9 in table 2 (0.102 g, 59% yield):

¹NMR (DMSO d₆): 10.30 (s, 1H), 8.46 (s, 1H), 7.72 (m, 1H), 7.18 (m, 2H), 6.79 (s, 1H), 6.77 (s, 1H), 6.75 (m, 1H), 4.35 (m, 2H), 3.90 (s, 3H), 3.83 (s, 2H), 3.53 (m, 2H), 2.91 (m, 2H), 2.56 (m, 2H), 2.32 (s, 3H).

MS (+ve ESI) 528 (M+H)⁺.

### EXAMPLE 10

### Preparation of Compound 10 in Table 3

### N-(2,3-difluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide

A solution of 2-(3-{[5-(2-chloroethoxy)-7-(2-methoxyethoxy)quinazolin-4-yl]amino}-1H-pyrazol-5-yl)-N-(2,3-difluorophenyl)acetamide (0.569 g, 11.0 mmol), 2-(methylamino)ethanol (0.375 g, 5.0 mmol) and potassium iodide (0.332 g, 2.0 mmol) in dimethylacetamide (4 ml) was heated at 90° C. for 6 hours. The mixture was allowed to cool to room temperature and then diluted with dichloromethane (40 ml) and the mixture stirred at room temperature for 30 minutes. The mixture was filtered and then purified directly by flash chromatography on silica eluting first with a mixture of dichloromethane:dimethylacetamide 95:5 followed by a mixture of dichloromethane:methanol 95:5 and finally with a mixture of dichloromethane:methanol:880-aqueous ammonia solution 100:8:1. Fractions containing product were combined and evaporated in vacuo to leave compound 10 in table 3 (0.12 g, 21% yield):

¹H-NMR (DMSO d₆ at 373° K): 12.05 (br s, 1H), 10.17 (s, 1H), 9.76 (s, 1H), 8.43 (s, 1H), 7.67 (m, 1H), 7.15 (m, 2H), 6.8 (s, 1H), 6.78 (s, 1H), 4.38 (t, 2H), 4.27 (dd, 2H), 3.82 (s, 2H), 3.73 (dd, 2H), 3.53 (t, 2H), 3.36 (s, 3H), 2.97 (t, 2H), 2.61 (t, 2H), 2.36 (s, 3H).

MS (+ve ESI): 572 (M+H)⁺

2-(3-{[5-(2-chloroethoxy)-7-(2-methoxyethoxy)quinazolin-4-yl]amino}-1H-pyrazol-5-yl)-N-(2,3-difluorophenyl)acetamide used as starting material was prepared as follows:

a) To a solution of [7-(benzyloxy)-5-hydroxy-4-oxoquinazolin-3(4H)-yl]methyl pivalate (15.28 g, 40 mmol) (see: Hennequin, Laurent Francois Andre; Ple, Patrick. Preparation of 4-anilinoquinazoline derivatives for the treatment of tumors. PCT Int. Appl. WO01/094341), 2-chloroethanol (3.38 g, 42 mmol) and triphenylphosphine (12.58 g, 48 mmol) in tetrahydrofuran (60 ml) was added a solution of di-tert-butylazodicarboxylate (11.04 g, 48 mmol) in tetrahydrofuran (40 ml) keeping the internal temperature below 35° C. The mixture was stirred at room temperature for 1.5 hours and then evaporated in vacuo. The residue was triturated with ethanol (150 ml) and then filtered. The residue was washed with cold ethanol and then left to dry in air overnight to leave [7-(benzyloxy)-5-(2-chloroethoxy)-4-oxoquinazolin-3(4H)-yl]methyl pivalate (14.15 g, 80% yield):

¹H-NMR (DMSO d₆): 8.32 (s, 1H), 7.53-7.31 (m, 5H), 6.81 (d, 1H), 6.73 (d, 1H), 5.81 (s, 2H), 5.25 (s, 2H), 4.33 (t, 2H), 3.95 (t, 2H), 1.11 (s, 9H).

b) A solution of [7-(benzyloxy)-5-(2-chloroethoxy)-4-oxoquinazolin-3(4H)-yl]methyl pivalate (14.1 g, 31.8 mmol) in trifluoroacetic acid (60 ml) was heated under reflux for 6 hours. The mixture was evaporated in vacuo and the residue co-evaporated twice with toluene. The residue was purified by flash silica chromatography eluting with a mixture of dichloromethane:methanol:880 ammonia solution 100:8:1 to give [5-(2-chloroethoxy)-7-hydroxy-4-oxoquinazolin-3(4H)-yl]methyl pivalate (9.54 g, 85% yield):

¹H-NMR (DMSO d₆): 10.61 (s, 1H), 8.25 (s, 1H), 6.54 (d, 1H), 6.49 (d, 1H), 5.79 (s, 2H), 4.28 (t, 2H), 3.95 (t, 2H), 1.11 (s, 9H).

MS (+ve ESI): 355/357 (M+H)⁺

c) A solution of di-tert-butylazodicarboxylate (7.39 g, 32.1 mmol) in tetrahydrofuran (20 ml) was added dropwise to a suspension of [5-(2-chloroethoxy)-7-hydroxy-4-oxoquinazolin-3(4H)-yl]methyl pivalate (9.52 g, 26.9 mmol), 2-methoxyethanol (2.14 g, 28.2 mmol) and triphenylphosphine (8.43 g, 32.2 mmol) in tetrahydrofuran (120 ml). The resulting solution was stirred at room temperature for 2 hours. The mixture was evaporated in vacuo and the residue triturated with ethanol (200 ml) and left to dry in air overnight to give [5-(2-chloroethoxy)-7-(2-methoxyethoxy)-4-oxoquinazolin-3(4H)-yl]methyl pivalate (5.33 g, 48% yield):

¹H-NMR (DMSO d₆): 8.32 (s, 1H), 6.72 (m, 1H), 6.65 (m, 1H), 5.81 (s, 2H), 4.34 (t, 2H), 4.23 (t, 2H), 3.95 (t, 2H), 3.68 (t, 2H), 3.31 (s, 3H), 1.11 (s, 9H).

MS (+ve ESI): 413/415 (M+H)⁺

d) A suspension of [5-(2-chloroethoxy)-7-(2-methoxyethoxy)-4-oxoquinazolin-3(4H)-yl]methyl pivalate (6.31 g, 15.3 mmol) in a solution of ammonia in methanol (7M, 160 ml) was stirred at 40° C. for 5.5 hours. The mixture was allowed to cool to room temperature and then stirred overnight. The mixture was evaporated in vacuo and the residue purified by flash silica chromatography eluting with a mixture of dichloromethane:methanol 9:1 to give 5-(2-chloroethoxy)-7-(2-methoxyethoxy)quinazolin-4(3H)-one (4.26 g, 93% yield):

¹H-NMR (DMSO d₆): 11.71 (s, 1H), 7.89 (d, 1H), 6.69 (d, 1H), 6.58 (d, 1H), 4.32 (t, 2H), 4.2 (m, 2H), 3.93 (t, 2H), 3.68 (m, 2H), 3.31 (s, 3H).

MS (+ve ESI): 299/301 (M+H)⁺

e) Phosphorus oxychloride (7.48 g, 48.8 mmol) was added to a suspension of 5-(2-chloroethoxy)-7-(2-methoxyethoxy)quinazolin-4(3H)-one (4.24 g, 14.2 mmol) and di-iso-propylethyl amine (12.6 g, 97.7 mmol) in 1,2-dichloroethane (225 ml) cooled in an ice-bath. The mixture was stirred at 0° C. for 15 minutes and then heated at 80° C. for 6 hours. The mixture was allowed to cool to room temperature and then stirred overnight. The mixture was evaporated in vacuo and the residue dissolved in dichloromethane (175 ml) and 2-(3-amino-1H-pyrazol-5-yl)-N-(2,3-difluorophenyl)acetamide (3.6 g, 14.3 mmol) followed by a solution of hydrogen chloride in dioxane (4M, 7.1 ml) added. The mixture was heated under reflux for 1 hour and then allowed to cool to room temperature. The mixture was filtered and the residue washed with dichloromethane and then acetone to give 2-(3-{[5-(2-chloroethoxy)-7-(2-methoxyethoxy)quinazolin-4-yl]amino}-1H-pyrazol-5-yl)-N-(2,3-difluorophenyl)acetamide as the hydrochloride salt (4.92 g, 61% yield over 2 steps):

¹H-NMR (DMSO d₆): 10.78 (s, 1H), 10.36 (s, 1H), 8.88 (s, 1H), 7.65 (m, 1H), 7.19 (m, 2H), 7.07 (dd, 2H), 6.79 (s, 1H), 4.7 (t, 2H), 4.3 (t, 2H), 4.17 (t, 2H), 3.91 (s, 2H), 3.73 (t, 2H), 3.32 (s, 3H).

MS (+ve ESI): 533/535 (M+H)⁺

### EXAMPLE 11

### Preparation of Compound 11 in Table 3-N-(2,3-difluorophenyl)-2-(3-{[5-{2-[ethyl(2-hydroxyethyl)amino]ethoxy}-7-(2-methoxyethoxy)quinazolin-4-yl]amino}-1H-pyrazol-5-yl)acetamide

Prepared in an analogous way to that described in example 10 in table 3 using 2-(ethylamino)ethanol (0.391 g, 4.4 mmol) and 2-(3-{[5-(2-chloroethoxy)-7-(2-methoxyethoxy)quinazolin-4-yl]amino}-1H-pyrazol-5-yl)-N-(2,3-difluorophenyl)acetamide (0.50 g, 0.88 mmol), after the reaction had finished the mixture was diluted with dichloromethane and the mixture purified directly by flash chromatography to give compound 11 in table 3 (0.21 g, 41% yield):

¹H-NMR (DMSO d₆ at 373° K): 12.1 (br s, 1H), 10.07 (br s, 1H), 9.75 (s, 1H), 8.45 (s, 1H), 7.67 (m, 1H), 7.15 (m, 2H), 6.8 (s, 1H), 6.75 (s, 1H), 4.39 (t, 2H), 4.27 (dt, 2H), 3.82 (s, 2H), 3.73 (m, 2H), 3.53 (t, 2H), 3.35 (s, 3H), 3.07 (t, 2H), 2.72 (m, 4H), 1.0 (t, 3H).

MS (+ve ESI): 586 (M+H)⁺

### EXAMPLE 12

### Preparation of Compound 12 in Table 3

### N-(2,3-difluorophenyl)-2-(3-{[5-{2-[(2S)-2-(hydroxymethyl)pyrrolidin-1-yl]ethoxy}-7-(2-methoxyethoxy)quinazolin-4-yl]amino}-1H-pyrazol-5-yl)acetamide

Prepared in an analogous way to that described in example 11 in table 3 using (2S)-pyrrolidin-2-ylmethanol (0.889 g, 8.8 mmol) and 2-(3-{[5-(2-chloroethoxy)-7-(2-methoxyethoxy)quinazolin-4-yl]amino}-1H-pyrazol-5-yl)-N-(2,3-difluorophenyl)acetamide (1.0 g, 1.8 mmol), after chromatography the crude product was then triturated with acetonitrile to give compound 12 in table 3 (0.49 g, 46% yield):

¹H-NMR (DMSO d₆ at 373° K): 10.05 (br s, 1H), 9.77 (s, 1H), 8.44 (s, 1H), 7.67 (t, 1H), 7.14 (m, 2H), 6.8 (s, 1H), 6.76 (s, 1H), 6.72-6.86 (m, 1H), 4.43 (m, 2H), 4.28 (dd, 2H), 3.82 (s, 2H), 3.72 (dd, 2H), 3.37-3.48 (m, 3H), 3.37 (s, 3H), 3.2 (m, 1H), 2.7 (broad, 1H), 1.82 (m, 1H), 1.65 (m, 2H), 1.57 (m, 1H).

MS (+ve ESI): 598 (M+H)⁺

### EXAMPLE 13

### Preparation of Compound 13 in Table 4

### N-(2,3-difluorophenyl)-2-{3-[(7-ethoxy-5-{2-[(2-hydroxyethyl)(methyl)amino]ethoxy}quinazolin-4-yl)amino]-1H-pyrazol-5-yl}acetamide

A mixture of 2-(3-{[5-(2-chloroethoxy)-7-ethoxyquinazolin-4-yl]amino}-1H-pyrazol-5-yl)-N-(2,3-difluorophenyl)acetamide (0.20 g, 0.40 mmol), 2-methylamino ethanol (0.119 g, 1.59 mmol) and potassium iodide (0.132 g, 0.80 mmol) in dimethylacetamide (2 ml) was heated at 90° C. for 3 hours. The mixture was cooled to room temperature and then poured into water (10 ml). The resultant solid was filtered and washed with water and dried in air. The crude product was purified by preparative HPLC (C18 silica column) eluting with a gradient of acetonitrile (0.2% trifluoroacetic acid) in water (0.2% trifluoroacetic acid). Fractions containing product were combined and made basic by the addition of solid sodium hydrogencarbonate and then concentrated in vacuo. The remaining mixture was extracted with a solution of iso-propyl alcohol in dichloromethane (10%). The extracts were dried over magnesium sulphate and evaporated. The residue was triturated with diethyl ether and dried in air to give compound 13 in table 4 (0.111 g, 52% yield):

¹NMR (DMSO d₆): 12.44 (br s, 1H), 10.35 (s, 1H), 10.26 (br s, 1H), 8.51 (s, 1H), 7.78 (m, 1H), 7.25 (m, 2H), 6.91 (br s, 1H), 6.82 (s, 1H), 6.78 (s, 1H), 4.69 (br s, 1H), 4.40 (m, 2H), 4.24 (q, 2H), 3.88 (s, 2H), 3.58 (m, 2H), 2.96 (m, 2H), 2.62 (m, 2H), 2.37 (s, 3H), 1.44 (t, 3H).

MS (+ve ESI) 542 (M+H)⁺.

2-(3-{[5-(2-chloroethoxy)-7-ethoxyquinazolin-4-yl]amino}-1H-pyrazol-5-yl)-N-(2,3-difluorophenyl)acetamide used as starting material was prepared as follows:

a) A solution of 5,7-difluoroquinazolin-4(3H)-one (2.0 g, 111.0 mmol) (see: Hennequin, Laurent Francois Andre; Ple, Patrick. Preparation of 4-anilinoquinazoline derivatives for the treatment of tumors. PCT Int. Appl. WO 01/094341) and sodium ethoxide (3.7 g, 54.4 mmol) in dimethylformamide was heated at 90° C. for 6 hours. The mixture was allowed to cool to room temperature and then poured into a solution of ammonium chloride (100 ml). The resultant precipitate was filtered, washed with water and then dried under high vacuum to give 5,7-diethoxyquinazolin-4(3H)-one (1.36 g, 53% yield):

MS (+ve ESI) 235 (M+H)⁺.

b) A solution of 5,7-diethoxyquinazolin-4(3H)-one (0.700 g, 2.99 mmol) and sodium methoxide (2.66 ml of 28% w/v solution in methanol, 13.8 mmol) in dimethylformamide (7 ml) was heated at 110° C. for 18 hours. The mixture was allowed to cool to room temperature and then made acidic by the addition of concentrated hydrochloric acid and then purified by preparative HPLC (C18 silica column) eluting with a gradient of acetonitrile (0.2% trifluoroacetic acid) in water (0.2% trifluoroacetic acid). Fractions containing product were combined and made basic by the addition of solid sodium hydrogencarbonate and then concentrated in vacuo. The resultant precipitate was filtered and washed with water followed by acetonitrile and then with diethyl ether and finally air dried to give 7-ethoxy-5-methoxyquinazolin-4(3H)-one (0.198 g, 30% yield):

¹NMR (DMSO d₆): 7.94 (s, 1H), 6.64 (s, 1H), 6.53 (s, 1H), 4.15 (q, 2H), 3.83 (s, 3H), 1.37 (t, 3H).

MS (+ve ESI) 221 (M+H)⁺.

c) Phosphorus oxychloride (4.76 g, 31.0 mmol) was added to a mixture of 7-ethoxy-5-methoxyquinazolin-4(3H)-one (2.0 g, 9.1 mmol) and di-iso-propylethyl amine (4.26 g, 33.0 mmol) in 1,2-dichloroethane (50 ml). The mixture was heated at 80° C. for 6 hours. The mixture was allowed to cool to room temperature and then evaporated in vacuo. The residue was partitioned between dichloromethane and a saturated solution of sodium hydrogencarbonate. The organic layer was separated, dried over magnesium sulphate and then evaporated in vacuo. The crude product was purified by flash chromatography on silica eluting with ethyl acetate to give 4-chloro-7-ethoxy-5-methoxyquinazoline (2.0 g, 92% yield):

MS (+ve ESI) 239/241 (M+H)⁺.

d) A mixture of 4-chloro-7-ethoxy-5-methoxyquinazoline (2.0 g, 8.4 mmol) and 2-(3-amino-1H-pyrazol-5-yl)-N-(2,3-difluorophenyl)acetamide (2.05 g, 8.13 mmol) in iso-propylalcohol (40 ml) was heated at 90° C. for 1 hour. The mixture was allowed to cool to room temperature and then diluted with diethyl ether (40 ml). The mixture was filtered and the residue washed with diethyl ether and then allowed to dry in air to give N-(2,3-difluorophenyl)-2-{3-[(7-ethoxy-5-methoxyquinazolin-4-yl)amino]-1H-pyrazol-5-yl}acetamide (3.76 g, 91% yield):

¹NMR (DMSO d₆): 10.71 (s, 1H), 10.36 (s, 1H), 8.83 (s, 1H), 7.69 (m, 1H), 7.20 (m, 2H), 7.01 (s, 1H), 6.93 (s, 1H), 6.72 (s, 1H), 4.24 (q, 2H), 4.16 (s, 3H), 3.92 (s, 2H), 1.42 (t, 3H).

MS (+ve ESI) 455 (M+H)⁺.

e) A mixture of N-(2,3-difluorophenyl)-2-{3-[(7-ethoxy-5-methoxyquinazolin-4-yl)amino]-1H-pyrazol-5-yl}acetamide (3.60 g, 7.34 mmol) and pyridine hydrochloride (5.2 g, 45.0 mmol) in pyridine (45 ml) was heated at 110° C. for 5 hours. The mixture was allowed to cool to room temperature and then poured into a saturated solution of sodium hydrogencarbonate (150 ml). The resultant solid was filtered and washed with water and then dried under high vacuum to give N-(2,3-difluorophenyl)-2-{3-[(7-ethoxy-5-hydroxyquinazolin-4-yl)amino]-1H-pyrazol-5-yl}acetamide (2.48 g, 77% yield):

MS (+ve ESI) 441 (M+H)⁺.

f) Di-tert-butylazodicarboxylate (0.502 g, 2.18 mmol) was added in one portion to a suspension of N-(2,3-difluorophenyl)-2-{3-[(7-ethoxy-5-hydroxyquinazolin-4-yl)amino]-1H-pyrazol-5-yl}acetamide (0.80 g, 1.82 mmol), 2-chloroethanol (0.205 g, 2.55 mmol) and triphenylphosphine (0.572 g, 2.18 mmol) in tetrahydrofuran (20 ml). The mixture was stirred for 1 hour at room temperature and then a further portion of triphenylphosphine (0.572 g, 2.18 mmol) and di-tert-butylazodicarboxylate (0.502 g, 2.18 mmol) were added. The mixture was stirred for 2 hours and then diluted with methyl tert-butylether (20 ml) and then filtered. The residue was washed with methyl tert-butylether and then dried in air to give 2-(3-{[5-(2-chloroethoxy)-7-ethoxyquinazolin-4-yl]amino}-1H-pyrazol-5-yl)-N-(2,3-difluorophenyl)acetamide (0.66 g, 72% yield):

MS (+ve ESI) 503 (M+H)⁺.

### EXAMPLE 14

### Preparation of Compound 14 in Table 4

### N-(2,3-difluorophenyl)-2-{3-[(7-ethoxy-5-{2-[(2S)-2-(hydroxymethyl)pyrrolidin-1-yl]ethoxy}quinazolin-4-yl)amino]-1H-pyrazol-5-yl}acetamide

Prepared in an analogous way to that described for example 13 in table 4 using (2S)-pyrrolidin-2-ylmethanol (0.161 g, 1.59 mmol) and 2-(3-{[5-(2-chloroethoxy)-7-ethoxyquinazolin-4-yl]amino}-1H-pyrazol-5-yl)-N-(2,3-difluorophenyl)acetamide (0.20 g, 0.40 mmol) to give compound 14 in table 4 (0.161 g, 71% yield):

¹NMR (DMSO d₆): 12.38 (s, 1H), 10.17 (s, 2H), 8.44 (s, 1H), 7.72 (m, 1H), 7.20 (m, 2H), 6.83 (s, 1H), 6.77 (s, 1H), 6.73 (s, 1H), 4.65 (br s, 1H), 4.39 (m, 1H), 4.34 (m, 1H), 4.18 (q, 2H), 3.84 (s, 2H), 3.39 (m, 2H), 3.34 (m, 1H), 3.17 (m, 1H), 2.76 (m, 1H), 2.56 (m, 1H), 2.32 (m, 1H), 1.77 (m, 1H), 1.58 (m, 3H), 1.39 (t, 3H).

MS (+ve ESI) 568 (M+H)⁺.

### EXAMPLE 15

### Preparation of Compound 15 in Table 4

### N-(2,3-difluorophenyl)-2-{3-[(7-ethoxy-5-{2-[(2-hydroxyethyl)(2-methoxyethyl)amino]ethoxy}quinazolin-4-yl)amino]-1H-pyrazol-5-yl}acetamide

Prepared in an analogous way to that described for example 13 in table 4 using 2-[(2-methoxyethyl)amino]ethanol (0.190 g, 1.60 mmol) and 2-(3-{[5-(2-chloroethoxy)-7-ethoxyquinazolin-4-yl]amino}-1H-pyrazol-5-yl)-N-(2,3-difluorophenyl)acetamide (0.20 g, 0.40 mmol) to give compound 15 in table 4 (0.127 g, 54% yield):

¹H-NMR (DMSO d₆): 12.37 (s, 1H), 10.18 (s, 2H), 8.44 (s, 1H), 7.72 (m, 1H), 7.18 (m, 2H), 6.83 (s, 1H), 6.76 (s, 1H), 6.73 (s, 1H), 4.61 (br s, 1H), 4.34 (t, 2H), 4.18 (q, 2H), 3.84 (s, 2H), 3.49 (m, 2H), 3.38 (m, 2H), 3.11 (s, 3H), 3.05 (t, 2H), 2.77 (t, 2H), 2.69 (t, 2H), 1.39 (t, 3H).

MS (+ve ESI) 586 (M+H)⁺.

### EXAMPLE 16

### Preparation of Compound 16 in Table 5

### 2-[(2-{[4-[5-{2-[(2,3-difluorophenyl)amino]-2-oxoethyl}-1H-pyrazol-3-yl)amino]-7-(2-methoxyethoxy)quinazolin-5-yl]oxy}ethyl)(methyl)amino]ethyl dihydrogen phosphate

Tetrazole (0.092 g, 1.3 mmol) was added to a solution of N-(2,3-difluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide (0.275 g, 0.44 mmol) in dimethylacetamide (4 ml) and the mixture stirred at room temperature for 30 seconds. Di-tert-butyl diethylphosphoramidite (0.436 g, 1.75 mmol) was added dropwise and the mixture was stirred at room temperature for 90 minutes. The mixture was cooled in an ice-bath and then an aqueous solution of hydrogen peroxide (30%, 2 mmol) was added. The mixture was allowed to warm to room temperature and stirred for 2 hours. The mixture was cooled in an ice-bath and the internal temperature maintained below 20° C. while an aqueous solution of sodium metabisulphite (10%, 2.2 mmol) was added. The mixture was stirred for 5 minutes and then made basic by the addition of an 20% aqueous solution of sodium hydrogencarbonate. The mixture was extracted with dichloromethane, the organic extracts were dried over sodium sulphate and then evaporated in vacuo. The residue was purified by flash silica chromatography eluting, over a gradient, with a mixture of dichloromethane:methanol:880 aqueous ammonia solution 100:5:0.5 to 100:15:1. Fractions containing product were combined and evaporated in vacuo to give di-tert-butyl 2-[(2-{[4-[(5-{2-[(2,3-difluorophenyl)amino]-2-oxoethyl}-1H-pyrazol-3-yl)amino]-7-(2-methoxyethoxy)quinazolin-5-yl]oxy}ethyl)(methyl)amino]ethyl phosphate. This intermediate was dissolved in dioxane (8 ml) and then a solution of hydrogen chloride in dioxane (4M, 0.66 ml, 2.6 mmol) was added and the mixture stirred for 18 hours at room temperature. The mixture was filtered and the residue was washed with dioxane and then with diethyl ether, the solid was dried under a nitrogen atmosphere and then at 60° C. under vacuum overnight to give compound 16 in table 5 as the di-hydrochloride salt (0.285 g, 86% yield):

¹H-NMR (DMSO-d₆): 10.34 (s, 1H), 8.79 (s, 1H), 7.67 (m, 1H), 7.19 (m, 2H), 7.01 (s, 2H), 6.77 (s, 1H), 4.82 (m, 2H), 4.31 (m, 2H), 4.26 (m, 2H), 3.91 (s, 2H), 3.79 (m, 2H), 3.74 (m, 2H), 3.55 (m, 2H), 3.34 (s, 3H), 2.99 (s, 3H).

MS (+ve ESI): 652 (M+H)⁺.

### EXAMPLE 17

### Preparation of Compound 17 in Table 5

### 2-[(2-{[4-[(5-{2-[(2,3-difluorophenyl)amino]-2-oxoethyl}-1H-pyrazol-3-yl)amino]-7-(2-methoxyethoxy)quinazolin-5-yl]oxy}ethyl)(ethyl)amino]ethyl dihydrogen phosphate

An analogous reaction to that described in example 16 but starting with N-(2,3-difluorophenyl)-2-(3-{[5-{2-[ethyl(2-hydroxyethyl)amino]ethoxy}-7-(2-methoxyethoxy)quinazolin-4-yl]amino}-1H-pyrazol-5-yl)acetamide (0.59 g, 1.0 mmol) yielded compound 17 in table 5 (0.25 g, 32% yield):

¹H-NMR (DMSO d₆): 10.35 (br s, 1H), 10.3 (s, 1H), 8.95 (br s, 1H), 8.65 (s, 1H), 7.67 (m, 1H), 7.16 (m, 2H), 6.92 (dd, 2H), 6.73 (s, 1H), 4.73 (t, 2H), 4.23 (m, 2H), 4.18 (m, 2H), 4.07 (m, 1H), 3.87 (s, 2H), 3.7 (m, 4H), 3.5 (t, 2H), 3.3 (s, 3H), 3.1 (t, 1H), 1.26 (t, 3H).

MS (+ve ESI): 666 (M+H)⁺

### EXAMPLE 18

### Preparation of Compound 18 in Table 5

### [(2S)-1-(2-{[4-[(5-{2-[(2,3-difluorophenyl)amino]-2-oxoethyl}-1H-pyrazol-3-yl)amino]-7-(2-methoxyethoxy)quinazolin-5-yl]oxy}ethyl)pyrrolidin-2-yl]methyl dihydrogen phosphate

An analogous reaction to that described in example 16 but starting with N-(2,3-difluorophenyl)-2-(3-{[5-{2-[(2S)-2-(hydroxymethyl)pyrrolidin-1-yl]ethoxy}-7-(2-methoxyethoxy)quinazolin-4-yl]amino}-1H-pyrazol-5-yl)acetamide (0.49 g, 0.82 mmol) yielded compound 18 in table 5 (0.305 g, 47% yield):

¹H-NMR (DMSO d₆): 10.65 (br s, 1H), 10.38 (s, 1H), 8.88 (s, 1H), 7.66 (t, 1H), 7.18 (m, 2H), 7.0 (dd, 2H), 6.72 (s, 1H), 4.75-4.9 (m, 2H), 4.15-4.3 (m, 4H), 3.7-4.05 (m, 8H), 3.3 (s, 3H), 2.2 (m, 1H), 1.75-2.1 (m, 3H).

MS (+ve ESI): 678 (M+H)⁺

### EXAMPLE 19

### Preparation of Compound 19 in Table 5

### 2-[[2-({4-[(5-{2-[(2,3-difluorophenyl)amino]-2-oxoethyl}-1H-pyrazol-3-yl)amino]-7-methoxyquinazolin-5-yl}oxy)ethyl](2-methoxyethyl)amino]ethyl dihydrogen phosphate

An analogous reaction to that described in example 16 but starting with N-(2,3-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide (0.35 g, 0.61 mmol) yielded compound 19 in table 5 (0.36 g, 78% yield):

¹NMR (DMSO d₆): 10.70 (br s, 1H), 10.40 (s, 1H), 8.88 (s, 1H), 7.67 (m, 1H), 7.21 (m, 2H), 7.12 (s, 1H), 7.05 (s, 1H), 6.75 (s, 1H), 4.86 (m, 2H), 4.31 (m, 2H), 3.99 (s, 3H), 3.94 (s, 2H), 3.89 (m, 2H), 3.80 (m, 2H), 3.65 (m, 2H), 3.60 (m, 2H), 3.27 (s, 3H).

MS (+ve ESI) 652 (M+H)⁺.

### EXAMPLE 20

### Preparation of Compound 20 in Table 5

### {(2S)-1-[2-({4-[(5-{2-[(2,3-difluorophenyl)amino]-2-oxoethyl}-1H-pyrazol-3-yl)amino]-7-methoxyquinazolin-5-yl}oxy)ethyl]pyrrolidin-2-yl}methyl dihydrogen phosphate

An analogous reaction to that described in example 16 but starting with N-(2,3-difluorophenyl)-2-{3-[(5-{2-[(2S)-2-(hydroxymethyl)pyrrolidin-1-yl]ethoxy}-7-methoxyquinazolin-4-yl)amino]-1H-pyrazol-5-yl}acetamide (0.35 g, 0.63 mmol) yielded compound 20 in table 5 (0.382 g, 82% yield):

¹NMR (DMSO d₆): 10.69 (br s, 1H), 10.40 (s, 1H), 8.88 (s, 1H), 7.66 (m, 1H), 7.19 (m, 2H), 7.10 (s, 1H), 7.06 (s, 1H), 6.75 (s, 1H), 4.91 (m, 1H), 4.86 (m, 1H), 4.34 (m, 1H), 4.25 (m, 1H), 4.02 (m, 1H), 3.99 (s, 3H), 3.93 (s, 2H), 3.86 (m, 4H), 2.22 (m, 1H), 2.04 (m, 1H), 1.96 (m, 1H), 1.83 (m, 1H).

MS (+ve ESI) 634 (M+H)⁺.

### EXAMPLE 21

### Preparation of Compound 21 in Table 5

### 2-[[2-({4-[(5-{2-[(2,3-difluorophenyl)amino]-2-oxoethyl}-1H-pyrazol-3-yl)amino]-7-methoxyquinazolin-5-yl}oxy)ethyl](methyl)amino]ethyl dihydrogen phosphate

An analogous reaction to that described in example 16 but starting with N-(2,3-difluorophenyl)-2-{3-[(5-{2-[(2-hydroxyethyl)(methyl)amino]ethoxy}-7-methoxyquinazolin-4-yl)amino]-1H-pyrazol-5-yl}acetamide (0.38 g, 0.72 mmol) yielded compound 21 in table 5 (0.32 g, 62% yield):

¹NMR (DMSO d₆): 10.67 (br s, 1H), 10.36 (s, 1H), 8.87 (s, 1H), 7.67 (m, 1H), 7.19 (m, 2H), 7.06 (s, 1H), 7.03 (s, 1H), 6.76 (s, 1H), 4.85 (m, 2H), 4.28 (m, 2H), 3.99 (s, 3H), 3.93 (s, 2H), 3.82 (m, 2H), 3.58 (m, 2H), 3.01 (s, 3H).

### EXAMPLE 22

### Preparation of Compound 22 in Table 5

### (2S)-2-{[2-({4-[(5-{2-[(3-fluorophenyl)amino]-2-oxoethyl}-1H-pyrazol-3-yl)amino]-7-methoxyquinazolin-5-yl}oxy)ethyl]amino}propyl dihydrogen phosphate

An analogous reaction to that described in example 16 but starting with N-(3-fluorophenyl)-2-(3-{[5-(2-{[(1S)-2-hydroxy-1-methylethyl]amino}ethoxy)-7-methoxyquinazolin-4-yl]amino}-1H-pyrazol-5-yl)acetamide (0.48 g, 0.94 mmol) yielded compound 22 in table 5 (0.556 g, 85% yield):

¹H-NMR (DMSO d₆+CD₃COOD): 8.87 (s, 11H), 7.64 (m, 1H), 7.34 (m, 2H), 7.05 (s, 2H), 6.87 (m, 1H), 6.74 (s, 1H); 4.78 (m, 2H), 4.12 (m, 2H), 4.26 (m, 2H), 3.98 (s, 3H); 3.86 (s, 2H), 3.79 (m, 2H), 3.66 (m, 1H), 3.56 (m, 2H), 1.35 (d, 3H).

MS (+ve ESI): 590 (M+H)⁺.

### EXAMPLE 23

### Preparation of Compound 23 in Table 6

### N-(3-fluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide

A mixture of 2-(3-{[5-(2-chloroethoxy)-7-(2-methoxyethoxy)quinazolin-4-yl]amino}-1-pyrazol-5-yl)-N-(3-fluorophenyl)acetamide (185 mg, 0.36 mmol), 2-(methylamino)ethanol (108 mg, 1.44 mmol) and potassium iodide (120 mg, 0.72 mmol) in dimethylacetamide (2 ml) were stirred and heated at 90° C. for 4 hours. The mixture was allowed to cool to room temperature and then purified directly by HPLC on C18 silica using a water/acetonitrile/TFA gradient to give compound 23 in table 6 (113 mg, 57% yield):

¹H-NMR (DMSO d₆): 12.38 (s, 1H), 10.41 (s, 1H), 10.29 (s, 1H), 8.45 (s, 1H), 7.61 (d, 1H), 7.39-7.31 (m, 2H), 6.91-6.85 (m, 2H), 6.77 (d, 2H), 4.65 (s, 1H), 4.36 (t, 2H), 4.25 (m, 2H), 3.75-3.71 (m, 4H), 3.52 (m, 2H), 3.34 (s, 3H), 2.91 (t, 2H), 2.56 (t, 2H), 2.32 (s, 3H).

MS (+ESI): 554 (M+H⁺)

2-(3-{[5-(2-Chloroethoxy)-7-(2-methoxyethoxy)quinazolin-4-yl]amino}-1H-pyrazol-5-yl)-N-(3-fluorophenyl)acetamide, used as starting material, was prepared as follows:

a) Phosphoryl chloride (532 μl, 5.70 mmol) was added dropwise, at room temperature, to a stirred mixture of 5-(2-chloroethoxy)-7-(2-methoxyethoxy)quinazolin-4(3H)-one (500 mg, 1.68 mmol) and N,N-diisopropylethylamine (992 μl, 5.70 mmol) in 1,2-dichloroethane (12 ml). The resulting orange solution was stirred and heated at 80° C. for 6.5 hours and then the bulk of the 1,2-dichloroethane was evaporated. The residue was taken up in dichloromethane, washed with saturated aqueous sodium hydrogen carbonate solution, dried over magnesium sulphate and evaporated to an orange gum. The gum was purified by chromatography on silica using ethyl acetate as eluent to give 4-chloro-5-(2-chloroethoxy)-7-(2-methoxyethoxy)quinazoline (402 mg, 76% yield) as a pale yellow solid.

MS (+ESI): 317 (M+H⁺).

b) A mixture of 4-chloro-5-(2-chloroethoxy)-7-(2-methoxyethoxy)quinazoline (2.01 g, 6.34 mmol), (3-amino-1H-pyrazol-5-yl)acetic acid (0.89 g, 6.31 mmol) and hydrogen chloride (1.6 ml of a 4M solution in 1,4-dioxane, 6.4 mmol) in dimethylacetamide (20 ml) were stirred at room temperature for 4 hours. The resulting thick suspension was added to water (100 ml) then 40% aqueous sodium hydroxide solution added to pH 12.2N Hydrochloric acid was then added to re-adjust the pH to 4.8. The resulting pale orange solid was filtered, washed with water and dried over phosphorus pentoxide under high vacuum to give (3-{[5-(2-chloroethoxy)-7-(2-methoxyethoxy)quinazolin-4-yl]amino}-1H-pyrazol-5-yl)acetic acid (2.45 g, 92% yield).

MS (+ESI): 422 (M+H⁺).

c) Pentafluorophenyl trifluoroacetate (212 mg, 0.76 mmol) was added dropwise, at room temperature, to a stirred suspension of (3-{[5-(2-chloroethoxy)-7-(2-methoxyethoxy)quinazolin-4-yl]amino}-1H-pyrazol-5-yl)acetic acid (160 mg, 0.38 mmol) and pyridine (61 μl, 0.76 mmol) in DMF (1.5 ml). The resulting solution was stirred for 15 minutes and then 3-fluoroaniline (73 μl, 0.76 mmol) was added. The reaction mixture was stirred at room temperature for 90 minutes and then heated at 90° C. for 2 hours. After cooling to room temperature the mixture was added to diethyl ether (15 ml). The resulting pale brown solid was filtered, washed with diethyl ether and then dried to leave 2-(3-{[5-(2-chloroethoxy)-7-(2-methoxyethoxy)quinazolin-4-yl]amino}-1H-pyrazol-5-yl)-N-(3-fluorophenyl)acetamide (185 mg, 95% yield).

MS (+ESI): 515 (M+H⁺).

### EXAMPLE 24

### Preparation of Compound 24 in Table 6

### N-(2,3-difluorophenyl)-2-(3-{[7-ethoxy-5-(2-{[(1S)-2-hydroxy-1-methylethyl]amino}ethoxy)quinazolin-4-yl]amino}-1H-pyrazol-5-yl)acetamide

A mixture of 2-(3-{[5-(2-chloroethoxy)-7-ethoxyquinazolin-4-yl]amino}-1H-pyrazol-5-yl)-N-(2,3-difluorophenyl)acetamide (0.25 g, 0.50 mmol), (2S)-2-aminopropan-1-ol (0.15 g, 2.0 mmol) and potassium iodide (0.165 g, 11.0 mmol) in dimethylacetamide (2.5 ml) was heated at 90° C. for 4 hours. The mixture was allowed to cool to room temperature and was then poured into water. The resultant precipitate was purified by preparative HPLC eluting with a gradient of acetonitrile (containing 0.2% trifluoroacetic acid) in water (containing 0.2% trifluoroacetic acid). Fractions containing product were combined, basified with sodium bicarbonate and then concentrated in vacuo. The mixture was extracted with a mixture of 10% iso-propanol in dichloromethane and the extracts dried over magnesium sulfate. The extracts were evaporated and the residue triturated with diethyl ether to give compound 24 in Table 6 (0.111 g, 52% yield):

¹H-NMR (DMSO d₆): 12.36 (s, 1H), 10.24 (s, 1H), 10.18 (s, 1H), 8.45 (s, 1H), 7.75-7.68 (m, 1H), 7.24-7.14 (m, 2H), 6.84 (s, 1H), 6.76 (s, 1H), 6.70 (s, 1H), 4.61 (s, 1H), 4.37-4.28 (m, 2H), 4.18 (q, 2H), 3.84 (s, 2H), ˜3.4-˜3.3 (m, peak under DMSO), 3.18-3.05 (m, 2H), 2.82-2.71 (m, 1H), 1.39 (t, 3H), 0.98 (d, 3H).

MS (+ESI): 542 (M+H⁺)

### EXAMPLE 25

### Preparation of Compound 25 in Table 6

### N-(2,3-difluorophenyl)-2-(3-{[7-ethoxy-5-(2-{[(1S)-1-(hydroxymethyl)propyl]amino}ethoxy)quinazolin-4-yl]amino}-1H-pyrazol-5-yl)acetamide

Prepared in an analogous procedure to that described in example 24 in Table 6 using (2S)-2-aminobutan-1-ol (0.142 g, 1.6 mmol), to give compound 25 in Table 6 (0.133 g, 60% yield):

¹H-NMR (DMSO d₆): 12.36 (br s, 1H), 10.27 (br s, 1H), 10.17 (s, 1H), 8.45 (s, 1H), 7.75-7.68 (m, 1H), 7.24-7.14 (m, 2H), 6.85 (br s, 1H), 6.76 (s, 1H), 6.70 (s, 1H), 4.55 (br s, 1H), 4.34-4.27 (m, 2H), 4.18 (q, 2H), 3.84 (br s, 2H), 3.47-3.89 (m, 1H), ˜3.4-˜3.3 (m, peak under DMSO), 3.18-3.07 (m, 2H), 1.39 (t, 3H), 0.87-0.77 (m, 2H).

MS (+ESI): 556 (M+H⁺)

### EXAMPLE 26

### Preparation of Compound 26 in Table 6

### N-(2,3-difluorophenyl)-2-(3-{[7-ethoxy-5-(2-{[(1S)-1(hydroxymethyl)-2-methylpropyl]amino}ethoxy)quinazolin-4-yl]amino}-1H-pyrazol-5-yl)acetamide

Prepared in an analogous procedure to that described in example 24 in Table 6 using (2S)-2-amino-3-methylbutan-1-ol (0.164 g, 1.6 mmol), to give compound 26 in Table 6 (0.148 g, 65% yield):

¹H-NMR (DMSO d₆): 12.35 (br s, 1H), 10.26 (br s, 1H), 10.17 (br s, 1H), 8.45 (s, 1H), 7.76-7.67 (m, 1H), 7.24-7.14 (m, 2H), 6.86 (br s, 1H), 6.76 (s, 1H), 6.70 (s, 1H), 4.51 (br s, 1H), 4.34-4.25 (m, 2H), 4.18 (q, 2H), 3.84 (br s, 2H), 3.52-3.43 (m, 1H), 3.40-˜3.3 (m, 1H peak partially under DMSO), 3.23-3.14 (m, 1H), 3.13-3.05 (m, 1H), 2.40-2.31 (m, 1H), 1.81-1.70 (m, 1H), 1.39 (t, 3H), 0.82 (d, 6H).

MS (+ESI): 570 (M+H⁺)

### EXAMPLE 27

### Preparation of Compound 27 in Table 6

### N-(2,3-difluorophenyl)-2-(3-{[7-ethoxy-5-(2-{[(1R)-2-hydroxy-1-(methoxymethyl)ethyl]amino}ethoxy)quinazolin-4-yl]amino}-1H-pyrazol-5-yl)acetamide

Prepared in an analogous procedure to that described in example 24 in Table 6 using (2R)-2-amino-3-methoxypropan-1-ol [Meyers, A. I.; Schmidt, Wolfgang; McKennon, Marc J. Asymmetric addition to chiral aromatic and unsaturated oxazolines using a novel chiral auxiliary. Synthesis (1993), (2), 250-62] (0.167 g, 1.6 mmol), to give compound 27 in Table 6 (0.124 g, 55% yield):

¹H-NMR (DMSO d₆): 12.36 (br s, 1H), 10.26 (br s, 1H), 10.18 (br s, 1H), 8.44 (s, 1H), 7.77-7.67 (m, 1H), 7.24-7.14 (m, 2H), 6.84 (br s, 1H), 6.76 (s, 1H), 6.70 (s, 1H), 4.65-4.56 (m, 1H), 4.35-4.26 (m, 2H), 4.18 (q, 2H), 3.84 (br s, 2H), 3.50-3.3 (m, 2H peak partially under DMSO), 3.22-3.10 (m, 5H), 2.82-2.74 (m, 1H), 2.51 (t, 3H).

MS (+ESI): 572 (M+H⁺)

### EXAMPLE 28

### Preparation of Compound 28 in Table 6

### 2-{3-[(5-{2-[(2-hydroxyethyl)(2-methoxyethyl)amino]ethoxy}-7-methoxyquinazolin-4-yl)amino]-1H-pyrazol-5-yl}-N-phenylacetamide

A mixture of 2-(3-{[5-(2-chloroethoxy)-7-methoxyquinazolin-4-yl]amino}-1H-pyrazol-5-yl)-N-phenylacetamide, 2-[(2-methoxyethyl)amino]ethanol (0.21 g, 1.8 mmol) and potassium iodide (0.14 g, 0.84 mmol) in dimethylacetamide (2 ml) was heated at 90° C. for 4 hours. The mixture was allowed to cool to room temperature and then purified directly by preparative HPLC eluting with a gradient of acetonitrile (containing 0.2% trifluoroacetic acid) in water (containing 0.2% trifluoroacetic acid). Fractions containing product were combined, basified with sodium bicarbonate and then extracted with dichloromethane. The extracts were dried over magnesium sulfate and then evaporated. The residue was triturated with diethyl ether to give compound 28 in Table 6 (0.118 g, 52% yield):

¹H-NMR (DMSO d₆): 12.38 (br s, 1H), 10.19 (br s, 1H), 8.45 (br s, 1H), 7.61 (d, 2H), 7.36-7.28 (m, 2H), 7.10-7.02 (m, 1H), 6.82 (br s, 1H), 6.79 (s, 1H), 6.74 (s, 1H), 4.62 (br s, 1H), 4.37-4.30 (m, 2H), 3.90 (s, 3H), 3.74 (br s, 2H), 3.53-3.46 (m, 2H), 3.41-3.34 (m, 2H), 3.11 (s, 2H), 3.06 (t, 2H), 2.77 (t, 2H), 2.69 (t, 2H).

MS (+ESI): 536 (M+H⁺)

2-(3-{[5-(2-chloroethoxy)-7-methoxyquinazolin-4-yl]amino}-1H-pyrazol-5-yl)-N-phenylacetamide, used as starting material, was prepared as follows:

a) A solution of di-tert-butylazodicarboxylate (3.08 g, 13.4 mmol) in tetrahydrofuran (10 ml) was added dropwise to a solution of [5-(2-chloroethoxy)-7-hydroxy-4-oxoquinazolin-3(4H)-yl]methyl pivalate (3.96 g, 11.2 mmol), methanol (0.39 g, 12.2 mmol) and triphenylphosphine (3.50 g, 13.4 mmol) in tetrahydrofuran (25 ml) and the mixture stirred at room temperature for 75 minutes. A further portion of triphenylphosphine (3.5 g, 13.4 mmol) was added followed by a solution of di-tert-butylazodicarboxylate (3.08 g, 13.4 mmol) in tetrahydrofuran (10 ml) and the mixture was stirred at room temperature for 90 minutes. A further portion of triphenylphosphine (3.5 g, 13.4 mmol) was added followed by a solution of di-tert-butylazodicarboxylate (3.08 g, 13.4 mmol) in tetrahydrofuran (10 ml) and the mixture was stirred at room temperature for 60 minutes. The mixture was evaporated and the residue taken up in ethyl acetate (50 ml) and then treated with hydrochloric acid (4N). The resultant precipitate was filtered and washed with ethyl acetate. The residue was dissolved in methanol and the mixture was then evaporated. The residue was partitioned between dichloromethane and a saturated solution of sodium bicarbonate. The organic phase was separated, dried over magnesium sulfate and evaporated. The residue was triturated with diethyl ether to leave [5-(2-chloroethoxy)-7-methoxy-4-oxoquinazolin-3(4H)-yl]methyl pivalate (3.0 g, 75% yield) which was used in the next step without further purification:

MS (+ESI): 369 (M+H⁺)

b) A mixture of [5-(2-chloroethoxy)-7-methoxy-4-oxoquinazolin-3(4H)-yl]methyl pivalate (3.0 g, 8.14 mmol) in a solution of ammonia in methanol (7N, 60 ml) was warmed to 50° C. to give a clear solution and then allowed to stir at room temperature for 24 hours. The mixture was diluted with methyl tert-butyl ether (60 ml) and then filtered. The residue was washed with methyl tert-butyl ether and then dried to give 5-(2-chloroethoxy)-7-methoxyquinazolin-4(3H)-one (1.62 g, 78% yield):

¹H-NMR (DMSO d₆): 11.73 (br s, 1H), 7.92 (s, 1H), 6.71 (d, 1H), 6.58 (d, 1H), 4.32 (t, 2H), 3.95 (t, 2H), 3.87 (s, 3H).

MS (+ESI): 255/257 (M+H⁺)

c) Phosphorus oxychloride (0.819 g, 5.34 mmol) was added dropwise to a stirred suspension of 5-(2-chloroethoxy)-7-methoxyquinazolin-4(3H)-one (0.40 g, 1.57 mmol) and N,N-di-iso-propylethylamine (0.689 g, 5.34 mmol) in 1,2-dichloroethane (10 ml). The mixture was heated at 80° C. for 5 hours. The mixture was evaporated and the residue taken up in dichloromethane and then washed with a saturated solution of sodium bicarbonate. The organic layer was separated, dried over magnesium sulfate and evaporated. The residue was purified by silica gel chromatography eluting with ethyl acetate to give 4-chloro-5-(2-chloroethoxy)-7-methoxyquinazoline (0.30 g, 70% yield):

MS (+ESI): 273/275/277 (M+H⁺)

d) A solution of hydrogen chloride in dioxane (4N, 0.27 ml) was added to a mixture of 4-chloro-5-(2-chloroethoxy)-7-methoxyquinazoline (0.30 g, 1.1 mmol) and (3-amino-1H-pyrazol-5-yl)acetic acid (0.155 g, 1.1 mmol) in dimethylacetamide (3 ml) and the mixture was stirred at room temperature for 5 hours. The mixture was poured into water (15 ml) and the mixture made basic (pH 12) with the addition of 40% sodium hydroxide solution. The clear solution was taken to pH4.8 by the addition of dilute hydrochloric acid. The resultant precipitate was filtered and the residue washed with water and then dried over phosphorus pentoxide under high vacuum to give (3-{[5-(2-chloroethoxy)-7-methoxyquinazolin-4-yl]amino}-1H-pyrazol-5-yl)acetic acid (0.362 g, 87% yield):

MS (+ESI): 378/380 (M+H⁺)

e) Pentafluorophenyl trifluoroacetate (0.237 g, 0.84 mmol) was added dropwise to a stirred suspension of (3-{[5-(2-chloroethoxy)-7-methoxyquinazolin-4-yl]amino}-1H-pyrazol-5-yl)acetic acid (0.16 g, 0.42 mmol) and pyridine (0.067 g, 0.84 mmol) in dimethylformamide (1.5 ml). The mixture was stirred at room temperature for 15 minutes and then aniline (0.079 g, 0.84 mmol) was added. The mixture was stirred at room temperature for 45 minutes and then a further portion of aniline (0.01 ml) was added and the mixture was then heated at 90° C. for 3 hours. The mixture was allowed to cool to room temperature and then diluted with diethyl ether (15 ml). The resultant precipitate was triturated with diethyl ether and dried in air to give 2-(3-{[5-(2-chloroethoxy)-7-methoxyquinazolin-4-yl]amino}-1H-pyrazol-5-yl)-N-phenylacetamide (0.193 g, 100% yield) which was used in the next step without further purification:

MS (+ESI): 453/455 (M+H⁺)

### EXAMPLE 29

### Preparation of Compound 29 in Table 6

### N-(2,4-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide

Prepared in an analogous procedure to that described in example 28 in Table 6 using 2-(3-{[5-(2-chloroethoxy)-7-methoxyquinazolin-4-yl]amino}-1H-pyrazol-5-yl)-N-(2,4-difluorophenyl)acetamide (0.15 g, 0.31 mmol), to give compound 29 in Table 6 (0.068 g, 39% yield):

¹H-NMR (DMSO d₆): 12.37 (br s, 1H), 10.18 (s, 1H), 9.99 (br s, 1H), 8.45 (s, 1H), 7.90-7.82 (m, 1H), 7.37-7.28 (m, 1H), 7.10-7.03 (m, 1H), 6.83 (br s, 1H), 6.79 (s, 1H), 6.75 (s, 1H), 4.61 (br s, 1H), 4.37-4.30 (m, 2H), 3.90 (s, 3H), 3.81 (br s, 2H), 3.53-3.45 (m, 2H), 3.41-3.34 (m, 2H), 3.11 (s, 3H), 3.08-3.02 (m, 2H), 2.77 (t, 2H), 2.69 (t, 2H).

MS (+ESI): 572 (M+H⁺)

2-(3-{[5-(2-chloroethoxy)-7-methoxyquinazolin-4-yl]amino}-1H-pyrazol-5-yl)-N-(2,4-difluorophenyl)acetamide, used as starting material, was prepared as follows:

a) Prepared in an analogous procedure to that described in example 28 (e) in Table 6 using 2,4-difluoroaniline (0.137 g, 1.1 mmol), to give 2-(3-{[5-(2-chloroethoxy)-7-methoxyquinazolin-4-yl]amino}-1H-pyrazol-5-yl)-N-(2,4-difluorophenyl)acetamide (0.15 g, 72% yield):

MS (+ESI): 489/491 (M+H⁺)

### EXAMPLE 30

### Preparation of Compound 30 in Table 6

### N-(3,5-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide

Prepared in an analogous procedure to that described in example 28 in Table 6 using 2-(3-{[5-(2-chloroethoxy)-7-methoxyquinazolin-4-yl]amino}-1H-pyrazol-5-yl)-N-(3,5-difluorophenyl)acetamide (0.15 g, 0.31 mmol), to give compound 30 in Table 6 (0.072 g, 41% yield):

¹H-NMR (DMSO d₆): 12.40 (br s, 1H), 10.59 (s, 1H), 10.19 (s, 1H), 8.45 (s, 1H), 7.38-7.30 (m, 2H), 6.96-6.87 (m, 1H), 6.83 (br s, 1H), 6.79 (s, 1H), 6.74 (s, 1H), 4.64-4.58 (m, 1H), 4.38-4.30 (m, 2H), 3.90 (s, 3H), 3.77 (br s, 2H), 3.52-3.45 (m, 2H), 3.41-3.34 (m, 2H), 3.11 (s, 3H), 3.08-3.02 (m, 2H), 2.77 (t, 2H), 2.69 (t, 2H).

MS (+ESI): 572 (M+H⁺)

2-(3-{[5-(2-chloroethoxy)-7-methoxyquinazolin-4-yl]amino}-1H-pyrazol-5-yl)-N-(3,5-difluorophenyl)acetamide, used as starting material, was prepared as follows:

a) Prepared in an analogous procedure to that described in example 28 (e) in Table 6 using 3,5-difluoroaniline (0.137 g, 1.1 mmol), to give 2-(3-{[5-(2-chloroethoxy)-7-methoxyquinazolin-4-yl]amino}-1H-pyrazol-5-yl)-N-(3,5-difluorophenyl)acetamide (0.15 g, 72% yield):

MS (+ESI): 489/491 (M+H⁺)

### EXAMPLE 31

### Preparation of Compound 31 in Table 6

### N-(2,5-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide

Prepared in an analogous procedure to that described in example 28 in Table 6 using 2-(3-{[5-(2-chloroethoxy)-7-methoxyquinazolin-4-yl]amino}-1H-pyrazol-5-yl)-N-(2,5-difluorophenyl)acetamide (0.18 g, 0.37 mmol), to give compound 31 in Table 6 (0.048 g, 23% yield):

¹H-NMR (DMSO d₆): 12.37 (br s, 1H), 10.19 (br s, 2H), 8.45 (s, 1H), 7.96-7.88 (m, 1H), 7.38-7.29 (m, 1H), 7.03-6.94 (m, 1H), 6.83 (br s, 1H), 6.79 (s, 1H), 6.75 (s, 1H), 4.61 (br s, 1H), 4.34 (t, 2H), 3.90 (s, 3H), 3.89-3.82 (m, 2H), 3.53-3.46 (m, 2H), 3.41-3.34 (m, 2H), 3.11 (s, 3H), 3.06 (t, 2H), 2.77 (t, 2H), 2.69 (t, 2H).

MS (+ESI): 572 (M+H⁺)

2-(3-{[5-(2-chloroethoxy)-7-methoxyquinazolin-4-yl]amino}-1H-pyrazol-5-yl)-N-(2,5-difluorophenyl)acetamide, used as starting material, was prepared as follows:

a) Prepared in an analogous procedure to that described in example 28 (e) in Table 6 using 2,5-difluoroaniline (0.137 g, 1.1 mmol), to give 2-(3-{[5-(2-chloroethoxy)-7-methoxyquinazolin-4-yl]amino}-1H-pyrazol-5-yl)-N-(2,5-difluorophenyl)acetamide (0.18 g, 87% yield):

MS (+ESI): 489/491 (M+H⁺)

### EXAMPLE 32

### Preparation of Compound 32 in Table 6

### N-(2,3-difluorophenyl)-2-(3-{[5-{2-[(2R)-2-(hydroxymethyl)pyrrolidin-1-yl]ethoxy}-7-(2-methoxyethoxy)quinazolin-4-yl]amino}-1H-pyrazol-5-yl)acetamide

Prepared in an analogous procedure to that described in example 10 in Table 3 using (2R)-pyrrolidin-2-ylmethanol (0.296 g, 2.93 mmol), to give compound 32 in Table 6 (0.243 g, 10% yield):

¹H-NMR (DMSO d₆): 12.1-12.0 (br s, 1H), 10.08 (s, 1H), 9.75 (s, 1H), 8.42 (s, 1H), 7.70-7.66 (m, 1H), 7.20-7.10 (m, 2H), 6.85-6.76 (m, 3H), 4.45-4.33 (m, 2H), 4.29-4.26 (m, 2H), 4.18-4.08 (br s, 1H), 3.86-3.78 (br s, 1H), 3.76-3.73 (m, 2H), 3.38 (s, 3H), 3.43-3.32 (m, 3H), 3.19-3.14 (m, 1H), 2.89-2.84 (m, 1H), 2.68-2.60 (m, 1H), 2.45-2.39 (m, 1H), 1.85-1.77 (m, 1H), 1.68-1.60 (m, 2H), 1.60-1.50 (m, 1H)

MS (+ESI): 598 (M+H⁺)

### EXAMPLE 33

### Preparation of Compound 33 in Table 6

### N-(4-chloro-2-fluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide

Prepared in an analogous procedure to that described in example 23 in Table 6 using 2-(3-{[5-(2-chloroethoxy)-7-(2-methoxyethoxy)quinazolin-4-yl]amino}-1H-pyrazol-5-yl)-N-(4-chloro-2-fluorophenyl)acetamide (0.19 g, 0.35 mmol) and 2-(methylamino)ethanol (0.104 g, 1.39 mmol), to give compound 33 in Table 6 (0.102 g, 50% yield):

¹H-NMR (DMSO d₆): 12.36 (s, 1H), 10.28 (s, 1H), 10.09 (br s, 1H), 8.44 (s, 1H), 8.1-7.92 (m, 1H), 7.53-7.45 (m, 1H), 7.30-7.23 (m, 1H), 6.85 (br s, 1H), 6.78 (s, 1H), 6.76 (s, 1H), 4.68-4.61 (m, 1H), 4.40-4.31 (m, 2H), 4.28-4.22 (m, 2H), 3.83 (br s, 2H), 3.74-3.69 (m, 2H), 3.56-3.49 (m, 2H), 3.34 (s, 3H), 2.94-2.88 (m, 2H), 2.56 (t, 2H), 2.32 (s, 3H).

MS (+ESI): 588/590 (M+H⁺)

2-(3-{[5-(2-chloroethoxy)-7-(2-methoxyethoxy)quinazolin-4-yl]amino}-1H-pyrazol-5-yl)-N-(4-chloro-2-fluorophenyl)acetamide, used as starting material, was prepared as follows:

a) Prepared in an analogous procedure to that described in example 23 (c) using 4-chloro-2-fluoroaniline (0.110 g, 0.76 mmol), to give 2-(3-{[5-(2-chloroethoxy)-7-(2-methoxyethoxy)quinazolin-4-yl]amino}-1H-pyrazol-5-yl)-N-(4-chloro-2-fluorophenyl)acetamide (0.19 g, 91% yield):

MS (+ESI): 549 (M+H⁺)

### EXAMPLE 34

### Preparation of Compound 34 in Table 6

### N-(3-chloro-2-fluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide

Prepared in an analogous procedure to that described in example 23 in Table 6 using 2-(3-{[5-(2-chloroethoxy)-7-(2-methoxyethoxy)quinazolin-4-yl]amino}-1H-pyrazol-5-yl)-N-(3-chloro-2-fluorophenyl)acetamide (0.20 g, 0.36 mmol) and 2-(methylamino)ethanol (0.109 g, 1.45 mmol), to give compound 34 in Table 6 (0.108 g, 50% yield):

¹H-NMR (DMSO d₆): 12.37 (s, 1H), 10.29 (s, 1H), 10.16 (br s, 1H), 8.45 (s, 1H), 7.88 (t, 1H), 7.35 (t, 1H), 7.20 (t, 1H), 6.86 (br s, 1H), 6.79 (s, 1H), 6.77 (s, 1H), 4.69-4.61 (m, 1H), 4.39-4.32 (m, 2H), 4.28-4.22 (m, 2H), 3.84 (br s, 2H), 3.75-3.69 (m, 2H), 3.56-3.49 (m, 2H), 3.34 (s, 3H), 2.91 (t, 2H), 2.57 (t, 2H), 2.32 (s, 3H).

MS (+ESI): 588/590 (M+H⁺)

2-(3-{[5-(2-chloroethoxy)-7-(2-methoxyethoxy)quinazolin-4-yl]amino}-1H-pyrazol-5-yl)-N-(3-chloro-2-fluorophenyl)acetamide, used as starting material, was prepared as follows:

a) Prepared in an analogous procedure to that described in example 23 (c) using 3-chloro-2-fluoroaniline (0.110 g, 0.76 mmol), to give 2-(3-{[5-(2-chloroethoxy)-7-(2-methoxyethoxy)quinazolin-4-yl]amino}-1H-pyrazol-5-yl)-N-(3-chloro-2-fluorophenyl)acetamide (0.20 g, 96% yield):

MS (+ESI): 549 (M+H⁺)

1. A compound of formula (I) 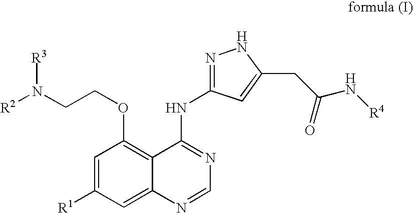
or a salt, ester or prodrug thereof;
wherein
R¹ is hydrogen or C₁₋₄alkoxy optionally substituted by C₁₋₄alkoxy;
R² is a group of formula (IA) wherein * is the point of attachment to formula (I); 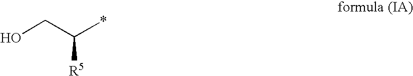
R³ is hydrogen or C₁₋₄alkyl optionally substituted by C₁₋₄alkoxy;
or R² and R³ together with the nitrogen atom to which they are attached form a ring of formula (IB) wherein * is the point of attachment to formula (I); 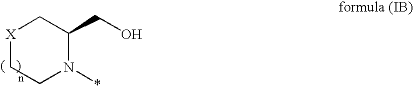
or R² and R³ together with the nitrogen atom to which they are attached form a ring of formula (IC) wherein * is the point of attachment to formula (I), provided that in this case, R¹ is C₂₋₄alkoxy optionally substituted by C₁₋₄alkoxy; 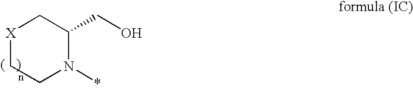
R⁴ is phenyl optionally substituted by 1 or 2 halo;
R⁵ is hydrogen or C₁₋₄alkyl optionally substituted by C₁₋₄alkoxy;
n is 0 or 1; and
X is CH₂, NH, N(C₁₋₄alkyl), O or S.

2. A compound according to claim 1, or a salt ester or prodrug thereof, wherein R² is 2-hydroxyethyl, (1S)-2-hydroxy-1-methylethyl, (1S)-2-hydroxy-1-ethylethyl, (1S)-2-hydroxy-1-isopropylethyl or (1S)-2-hydroxy-1-(methoxymethyl)ethyl.

3. A compound according to claim 2, or a salt ester or prodrug thereof, wherein R² is 2-hydroxyethyl or (1S)-2-hydroxy-1-methylethyl.

4. A compound according to claim 1, or a salt ester or prodrug thereof, wherein R³ is hydrogen, methyl, ethyl or methoxyethyl.

5. A compound according to claim 1, or a salt ester or prodrug thereof, wherein R² and R³ together with the nitrogen atom to which they are attached form: 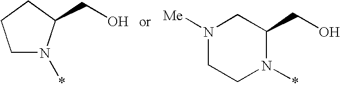
where * is the point of attachment to formula (I); or R² and R³ together with the nitrogen atom to which they are attached form: 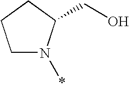
where * is the point of attachment to formula (I) and provided that, in this case, R¹ is ethoxy or methoxyethoxy.

6. A compound according to claim 5, or a salt ester or prodrug thereof, wherein R² and R³ together with the nitrogen atom to which they are attached form: 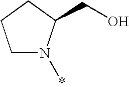
where * is the point of attachment to formula (I); or R² and R³ together with the nitrogen atom to which they are attached form: 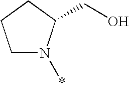
where * is the point of attachment to formula (I) and provided that, in this case, R¹ is ethoxy or methoxyethoxy.

7. A compound according to claim 6, or a salt ester or prodrug thereof, wherein R² and R³ together with the nitrogen atom to which they are attached form: 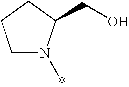
where * is the point of attachment to formula (I).

8. A compound according to claim 1, of formula (I′) 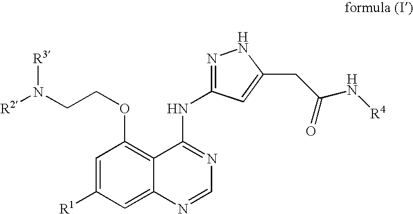
or a salt thereof;
wherein
R¹ is hydrogen or C₁₋₄alkoxy optionally substituted by C₁₋₄alkoxy;
R^{2′} is a group of formula (IA′) wherein * is the point of attachment to formula (I′); 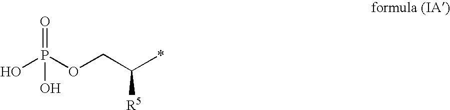
R^{3′} is hydrogen or C₁₋₄alkyl optionally substituted by C₁₋₄alkoxy;
or R^{2′} and R^{3′} together with the nitrogen atom to which they are attached form a ring of formula (IB′) wherein * is the point of attachment to formula (I′); 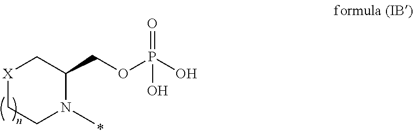
or R^{2′} and R^{3′} together with the nitrogen atom to which they are attached form a ring of formula (IC′) wherein * is the point of attachment to formula (I′), provided that, in this case, R¹ is C₂₋₄alkoxy optionally substituted by C₁₋₄alkoxy; 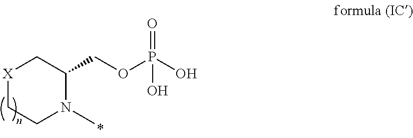
R⁴ is phenyl optionally substituted by 1 or 2 halo;
R⁵ is hydrogen or C₁₋₄alkyl optionally substituted by C₁₋₄alkoxy;
n is 0 or 1; and
X is CH₂, NH, N(C₁₋₄alkyl), O or S.

9. A compound according to claim 8, or a salt thereof, wherein R^{2′} is 2-phosphonooxyethyl, (1S)-2-phosphonooxy-1-methylethyl, (1S)-2-phosphonooxy-1-ethylethyl, (1S)-2-phosphonooxy-1-isopropylethyl or (1S)-2-phosphonooxy-1-(methoxymethyl)ethyl.

10. A compound according to claim 9, or a salt thereof, wherein R^{2′} is 2-phosphonooxyethyl or (1S)-2-phosphonooxy-1-methylethyl.

11. A compound according to claim 8, or a salt thereof, wherein R^{3′} is hydrogen, methyl, ethyl or methoxyethyl.

12. A compound according to claim 8, or a salt thereof, wherein R^{2′} and R^{3′} together with the nitrogen atom to which they are attached form: 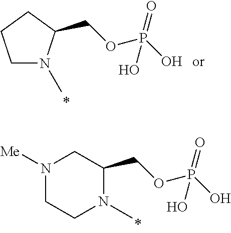
where * is the point of attachment to formula (I); or R^{2′} and R^{3′} together with the nitrogen atom to which they are attached form: 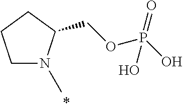
where * is the point of attachment to formula (I) and provided that in this case R¹ is ethoxy or methoxyethoxy.

13. A compound according to claim 12, or a salt thereof, wherein R^{2′} and R^{3′} together with the nitrogen atom to which they are attached form: 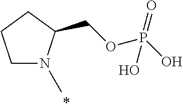
where * is the point of attachment to formula (I); or R^{2′} and R^{3′} together with the nitrogen atom to which they are attached form: 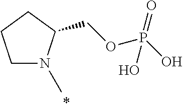
where * is the point of attachment to formula (I) and provided that, in this case, R¹ is ethoxy or methoxyethoxy.

14. A compound according to claim 13, or a salt thereof, wherein R^{2′} and R^{3′} together with the nitrogen atom to which they are attached form: 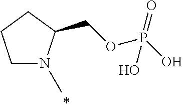
where * is the point of attachment to formula (I).

15. A compound according to claim 1, or a salt ester or prodrug thereof, wherein R¹ is C₁₋₄alkoxy optionally substituted by methoxy.

16. A compound according to claim 15, or a salt ester or prodrug thereof, wherein R¹ is methoxy, ethoxy or methoxyethoxy.

17. A compound according to claim 1, or a salt ester or prodrug thereof, wherein R⁴ is phenyl optionally substituted by 1 or 2 fluoro or chloro.

18. A compound according to claim 17, or a salt ester or prodrug thereof, wherein R⁴ is phenyl, 3-fluorophenyl, 2,3-difluorophenyl, 2,4-difluorophenyl, 2,5-difluorophenyl, 2-fluoro-3-chlorophenyl or 2-fluoro-4-chlorophenyl.

19. A compound according to claim 18, or a salt ester or prodrug thereof, wherein R⁴ is 3-fluorophenyl or 2,3-difluorophenyl.

20. A compound according to claim 1 selected from:
N-(3-fluorophenyl)-2-{3-[(5-{2-[(2-hydroxyethyl)(methyl)amino]ethoxy}-7-methoxyquinazolin-4-yl)amino]-1H-pyrazol-5-yl}acetamide;
N-(3-fluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(3-fluorophenyl)-2-{3-[(5-{2-[(2S)-2-(hydroxymethyl)pyrrolidin-1-yl]ethoxy}-7-methoxyquinazolin-4-yl)amino]-1H-pyrazol-5-yl}acetamide;
N-(3-fluorophenyl)-2-(3-{[5-(2-{[(1S)-2-hydroxy-1-methylethyl]amino}ethoxy)-7-methoxyquinazolin-4-yl]amino}-1H-pyrazol-5-yl)acetamide;
N-(2,3-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(2,3-difluorophenyl)-2-{3-[(5-{2-[ethyl(2-hydroxyethyl)amino]ethoxy}-7-methoxyquinazolin-4-yl)amino]-1H-pyrazol-5-yl}acetamide;
N-(2,3-difluorophenyl)-2-{3-[(5-{2-[(2S)-2-(hydroxymethyl)pyrrolidin-1-yl]ethoxy}-7-methoxyquinazolin-4-yl)amino]-1H-pyrazol-5-yl}acetamide;
N-(2,3-difluorophenyl)-2-{3-[(5-{2-[(2S)-2-(hydroxymethyl)-4-methylpiperazin-1-yl]ethoxy}-7-methoxyquinazolin-4-yl)amino]-1H-pyrazol-5-yl}acetamide;
N-(2,3-difluorophenyl)-2-{3-[(5-{2-[(2-hydroxyethyl)(methyl)amino]ethoxy}-7-methoxyquinazolin-4-yl)amino]-1H-pyrazol-5-yl}acetamide;
N-(2,3-difluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide;
N-(2,3-difluorophenyl)-2-(3-{[5-{2-[ethyl(2-hydroxyethyl)amino]ethoxy}-7-(2-methoxyethoxy)quinazolin-4-yl]amino}-1H-pyrazol-5-yl)acetamide;
N-(2,3-difluorophenyl)-2-(3-{[5-{2-[(2S)-2-(hydroxymethyl)pyrrolidin-1-yl]ethoxy}-7-(2-methoxyethoxy)quinazolin-4-yl]amino}-1H-pyrazol-5-yl)acetamide;
N-(2,3-difluorophenyl)-2-{3-[(7-ethoxy-5-{2-[(2-hydroxyethyl)(methyl)amino]ethoxy}quinazolin-4-yl)amino]-1H-pyrazol-5-yl}acetamide;
N-(2,3-difluorophenyl)-2-{3-[(7-ethoxy-5-{2-[(2S)-2-(hydroxymethyl)pyrrolidin-1-yl]ethoxy}quinazolin-4-yl)amino]-1H-pyrazol-5-yl}acetamide;
N-(2,3-difluorophenyl)-2-{3-[(7-ethoxy-5-{2-[(2-hydroxyethyl)(2-methoxyethyl)amino]ethoxy}quinazolin-4-yl)amino]-1H-pyrazol-5-yl}acetamide;
N-(3-fluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide;
N-(2,3-difluorophenyl)-2-(3-{[7-ethoxy-5-(2-{[(1S)-2-hydroxy-1-methylethyl]amino}ethoxy)quinazolin-4-yl]amino}-1H-pyrazol-5-yl)acetamide;
N-(2,3-difluorophenyl)-2-(3-{[7-ethoxy-5-(2-{[(1S)-1-(hydroxymethyl)propyl]amino}ethoxy)quinazolin-4-yl]amino}-1H-pyrazol-5-yl)acetamide;
N-(2,3-difluorophenyl)-2-(3-{[7-ethoxy-5-(2-{[(1S)-1-(hydroxymethyl)-2-methylpropyl]amino}ethoxy)quinazolin-4-yl]amino}-1H-pyrazol-5-yl)acetamide;
N-(2,3-difluorophenyl)-2-(3-{[7-ethoxy-5-(2-{[(1R)-2-hydroxy-1-(methoxymethyl)ethyl]amino}ethoxy)quinazolin-4-yl]amino}-1H-pyrazol-5-yl)acetamide;
2-{3-[(5-{2-[(2-hydroxyethyl)(2-methoxyethyl)amino]ethoxy}-7-methoxyquinazolin-4-yl)amino]-1H-pyrazol-5-yl}-N-phenylacetamide;
N-(2,4-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(3,5-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(2,5-difluorophenyl)-2-{3-[(5-{2-[(2-hydroxyethyl)(2-methoxyethyl)amino]ethoxy}-7-methoxyquinazolin-4-yl)amino]-1H-pyrazol-5-yl}acetamide;
N-(2,3-difluorophenyl)-2-(3-{[5-{2-[(2R)-2-(hydroxymethyl)pyrrolidin-1-yl]ethoxy}-7-(2-methoxyethoxy)quinazolin-4-yl]amino}-1H-pyrazol-5-yl)acetamide;
N-(4-chloro-2-fluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide; or
N-(3-chloro-2-fluorophenyl)-2-(3-{[5-{2-[(2-hydroxyethyl)(methyl)amino]ethoxy}-7-(2-methoxyethoxy)quinazolin-4-yl]amino}-1H-pyrazol-5-yl)acetamide;
2-[(2-{[4-[(5-{2-[(2,3-difluorophenyl)amino]-2-oxoethyl}-1H-pyrazol-3-yl)amino]-7-(2-methoxyethoxy)quinazolin-5-yl]oxy}ethyl)(methyl)amino]ethyl dihydrogen phosphate;
2-[(2-{[4-[(5-{2-[(2,3-difluorophenyl)amino]-2-oxoethyl}-1H-pyrazol-3-yl)amino]-7-(2-methoxyethoxy)quinazolin-5-yl]oxy}ethyl)(ethyl)amino]ethyl dihydrogen phosphate;
[(2S)-1-(2-{[4-[(5-{2-[(2,3-difluorophenyl)amino]-2-oxoethyl}-1H-pyrazol-3-yl)amino]-7-(2-methoxyethoxy)quinazolin-5-yl]oxy}ethyl)pyrrolidin-2-yl]methyl dihydrogen phosphate;
2-[[2-({4-[(5-{2-[(2,3-difluorophenyl)amino]-2-oxoethyl}-1H-pyrazol-3-yl)amino]-7-methoxyquinazolin-5-yl}oxy)ethyl](2-methoxyethyl)amino]ethyl dihydrogen phosphate;
{(2S)-1-[2-({4-[(5-{2-[(2,3-difluorophenyl)amino]-2-oxoethyl}-1H-pyrazol-3-yl)amino]-7-methoxyquinazolin-5-yl}oxy)ethyl]pyrrolidin-2-yl}methyl dihydrogen phosphate;
2-[[2-({4-[(5-{2-[(2,3-difluorophenyl)amino]-2-oxoethyl}-1H-pyrazol-3-yl)amino]-7-methoxyquinazolin-5-yl}oxy)ethyl](methyl)amino]ethyl dihydrogen phosphate; and
(2S)-2-{[2-({4-[(5-{2-[(3-fluorophenyl)amino]-2-oxoethyl}-1H-pyrazol-3-yl)amino]-7-methoxyquinazolin-5-yl}oxy)ethyl]amino}propyl dihydrogen phosphate;
or a pharmaceutically acceptable salt thereof.

21. A pharmaceutical composition comprising a compound of formula (I), or a pharmaceutically acceptable salt, ester or prodrug thereof as defined in claim 1 in association with a pharmaceutically acceptable diluent or carrier.

22. (canceled)

23. A method of treating cancer which comprises administering a compound of formula (I) or a pharmaceutically acceptable salt ester or prodrug thereof as claimed in claim 1.

24. A method of treating hyperproliferative diseases which comprises administering a compound of formula (I), or a pharmaceutically acceptable salt ester or prodrug thereof as claimed in claim 1.

25. Method according to claim 24 wherein the hyperproliferative disease is any one of colorectal, breast, lung, prostate, bladder, renal or pancreatic cancer or leukaemia or lymphoma.

26. A method of treating a human suffering from a disease in which the inhibition of one or more Aurora kinases is beneficial, comprising the steps of administering to a person in need thereof a therapeutically effective amount of a compound as defined in claim 1 or a pharmaceutically acceptable salt thereof.

27. (canceled)

28. A process for the preparation of a compound of formula (I) or a salt, ester or prodrug thereof, as claimed in claim 1, which process comprises reacting a compound of formula (II) where L¹ is a leaving group: 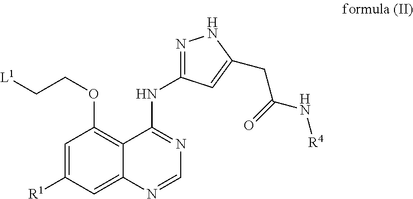
with an amine of formula (III): 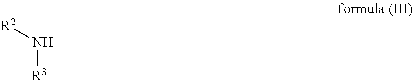
where R¹, R², R³ and R⁴ are as defined in claim 1 and thereafter if necessary:
i) converting a compound of formula (I) into another compound of formula (I);
ii) removing any protecting groups; and/or
iii) forming a salt, ester or prodrug.

29. A process for the preparation of a compound of formula (I′) or a salt thereof, as claimed in claim 8, which process comprises phosphorylation of a compound of formula (I): 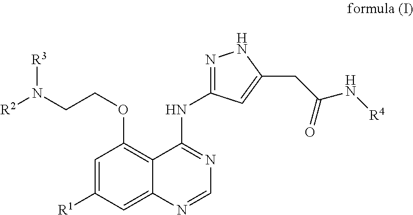
or a salt ester or prodrug thereof;
wherein
R¹ is hydrogen or C₁₋₄alkoxy optionally substituted by C₁₋₄alkoxy:
R² is a group of formula (IA) wherein * is the point of attachment to formula (I); 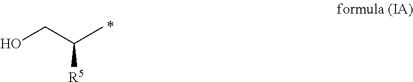
R³ is hydrogen or C₁₋₄alkyl optionally substituted by C₁₋₄alkoxy;
or R² and R³ together with the nitrogen atom to which they are attached form a ring of formula (IB) wherein * is the point of attachment to formula (I): 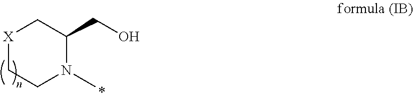
or R² and R³ together with the nitrogen atom to which they are attached form a ring of formula (IC) wherein * is the point of attachment to formula (I) provided that in this case, R¹ is C₂₋₄alkoxy optionally substituted by C₁₋₄alkoxy; 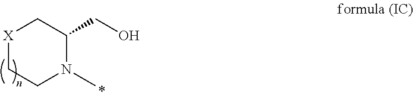
R⁴ is phenyl optionally substituted by 1 or 2 halo;
R⁵ is hydrogen or C₁₋₄alkyl optionally substituted by C₁₋₄alkoxy:
n is 0 or 1; and
X is CH₂ NH N(C₁₋₄alkyl), O or S;
and thereafter if necessary:
i) converting a compound of the formula (I′) into another compound of the formula (I′);
ii) removing any protecting groups; and/or
iii) forming a salt.
